(19) United States
(12) Patent Application Publication
(10) Pub. No.: US 2023/0263832 A1
(43) Pub. Date: Aug. 24, 2023

(54) TARGETED DRUG DELIVERY TO SITES OF INTRAVASCULAR OCCLUSION

(71) Applicant: Children's Medical Center Corporation, Boston, MA (US)

(72) Inventors: Shin-Young Park, Waltham, MA (US); Leslie E. Silberstein, Miami, FL (US); Jina Ryu, Seoul (KR); Min Liu, Brighton, MA (US)

(73) Assignee: Children's Medical Center Corporation, Boston, MA (US)

(21) Appl. No.: 18/015,209

(22) PCT Filed: Jul. 9, 2021
(86) PCT No.: PCT/US2021/041067

§ 371 Date: Jan. 9, 2023

Related U.S. Application Data
(60) Provisional application No. 63/050,477, filed on Jul. 10, 2020.

(51) Int. Cl. A61K 35/18; A61P 7/02; A61K 39/395; A61K 38/48; A61K 31/4709; A61K 31/4439; A61K 38/58; A61K 31/506

(52) U.S. Cl. CPC A61K 35/18; A61P 7/02; A61K 39/3955; A61K 38/482; A61K 31/4709; A61K 31/4439; A61K 38/58; A61K 31/506; C12Y 304/21068; A61K 2039/505

(57) ABSTRACT

Provided herein, in some aspects, are compositions comprising vasoocclusion-inhibiting agents encapsulated in RBCs and uses thereof for treating blood vessel occlusion (e.g., in Sickle Cell Disease).

### RELATED APPLICATIONS

This application claims the benefit under 35 U.S.C. § 119(e) of U.S. provisional application No. 63/050,477, filed Jul. 10, 2020, which is incorporated by reference herein in its entirety.

### GOVERNMENT SUPPORT

This invention was made with government support under Grant No. P01HL095489 awarded by the National Institutes of Health. The Government has certain rights in the invention.

### BACKGROUND

Sickle cell disease (SCD) is the most common genetic disorder with high morbidity and mortality. The polymerization of mutated sickle hemoglobin in the red blood cell (RBC) leads to the sickle red cell shape change, cell membrane abnormalities, hemolysis, and the entrapment of red cells in the micro-circulation, leading to the formation of intravascular RBC aggregates. These aggregates cause obstruction in blood vessels which produce symptoms in patients, and can lead to multi-organ damage. Extensive efforts are focused on understanding and treating the events which occur in the blood vessels of multiple organs in SCD.

### SUMMARY

Some aspects of the present disclosure provide a composition comprising a red blood cell (RBC) and a vasoocclusion-inhibiting agent wherein the vasoocclusion-inhibiting agent is encapsulated in the RBC.

In some embodiments, the vasoocclusion-inhibiting agent is an anti-adhesion agent.

In some embodiments, the anti-adhesion agent is an anti-P-selectin agent, Rivipansel, an anti-selectin aptamer, or an αvβ3 integrin inhibitor. In some embodiments, the anti-P-selectin agent is an anti-P-selectin antibody. The anti-P-selectin antibody can be a polyclonal antibody or a monoclonal antibody.

In some embodiments, the anti-P-selectin antibody is Crizanlizumab.

In some embodiments, the vasoocclusion-inhibiting agent is a tissue plasminogen activator. In some embodiments, the tissue plasminogen activator is Alteplase, Reteplase or Tenecteplase.

In some embodiments, the vasoocclusion-inhibiting agent is an anti-coagulant agent. In some embodiments, the anti-coagulant agent is a direct thrombin inhibitor. In some embodiments, the direct thrombin inhibitor is Argatroban, Dabigatrin, or Lepirudin.

In some embodiments, the vasoocclusion-inhibiting agent is an anti-inflammatory agent. In some embodiments, the anti-inflammatory agent is an endothelin antagonist. In some embodiments, the endothelin antagonist is Bosentan.

In some embodiments, the vasoocclusion-inhibiting agent is a modulator of ischaemia-reperfusion and oxidative stress.

In some embodiments, the vasoocclusion-inhibiting agent is an anti-platelet agent.

In some embodiments, the vasoocclusion-inhibiting agent is an agent that counteract free hemoglobin, heme, or iron.

In some embodiments, the vasoocclusion-inhibiting agent is encapsulated in the RBC by ex vivo electroporation. In some embodiments, the vasoocclusion-inhibiting agent is encapsulated in the RBC by endocytosis. In some embodiments, the vasoocclusion-inhibiting agent is encapsulated in the RBC by cell-penetrating peptide (CPP)-mediated internalization.

In some embodiments, the RBC is an autologous RBC. In some embodiments, the RBC is an allogenic RBC.

In some embodiments, the vasoocclusion-inhibiting agent is delivered to a site of a blood vessel occlusion. In some embodiments, the vasoocclusion-inhibiting agent is released at the site of a blood vessel occlusion.

In some embodiments, the blood vessel occlusion is caused by sickle cell disease.

In some embodiments, the blood vessel occlusion comprises a RBC aggregate.

In some embodiments, the blood vessel occlusion comprises a heterocellular aggregate.

In some embodiments, the heterocellular aggregate comprises a RBC(s), a white blood cell(s) (WBC(s)), and a platelet(s).

In some embodiments, the composition further comprising a pharmaceutically acceptable carrier.

Other aspects of the present disclosure provide methods of treating a blood vessel occlusion in a subject, the method comprising administering to the subject in need thereof an effective amount of the composition described herein.

In some embodiments, the blood vessel occlusion is caused by sickle cell disease.

In some embodiments, the composition is administered intravenously. In some embodiments, the composition is administered once. In some embodiments, the composition is administered repeatedly.

In some embodiments, the subject is a mammal. In some embodiments, the mammal is a human.

In some embodiments, the effective amount of the composition reduces the size of the occlusion by 10%, 20%, 30%, 40%, 50%, 60%, 70%, 80%, 90%, or 100%.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. The patent or application file contains at least one drawing executed in color. Copies of this patent or patent application publication with color drawing(s) will be provided by the Office upon request and payment of the necessary fee. In the drawings:

FIG. 1: Transfused RBCs localize in various organs in SCD mice. Fresh RBCs from wild type (WT) mouse were used for CFSE-labeling. One billion RBCs were incubated with 20 μM of CFSE for 12 min at 37° C. Then 40 million CFSE-labeled RBCs in phosphate buffered saline (PBS) were transfused to SCD mice by i.v. injection. After injecting CFSE-labeled RBCs intravenously into SS mice, the SS mice were euthanized on 24 h and perfused intracardially with PBS, followed by harvesting organs. Single cell suspension from different organs were stained with BV605-labeled Ter119 antibodies, followed by flow cytometry. The frequencies of CFSE-labeled RBCs are shown in the gates. Transfused RBC survival (%)=frequency of transfused RBCs on indicated time/frequency of transfused RBCs at injection×100.

FIG. 2: Transfused RBCs localize at the sites of RBC aggregates in SCD mouse bone marrow and spleen. Fresh RBCs from WT mouse were used for CFSE-labeling. One billion RBCs were incubated with 20 μM of CFSE for 12 min at 37° C. Then 40 million CFSE-labeled RBCs were transfused to SCD mice by i.v. injection. From transcardially PBS-perfused mice 24 h after injection, organs (bone marrow, spleen and liver) were harvested and fixed by PLP, followed by cryosectioning and immunofluorescent staining with antibodies against Ter119 (for RBC marker), Endoglin (for sinusoidal vasculature), and DAPI (for nucleus). Stained slides with fluorescent dye-labeled secondary antibodies were scanned on laser scan cytometry (LaSC), followed by quantitative imaging analysis (iCys software). Arrows indicate CFSE-labeled RBCs (Park et al. 2020).

FIG. 3: In SCD mouse liver sinusoids, transfused RBCs localize at the sites of RBC aggregates. LaSC analysis of harvested organs after transfusion of CFSE-labeled RBC, was performed as described in FIG. 2 legend.

FIGS. 4A to 4B: Transferrin proteins were encapsulated into RBCs by electroporation. (FIG. 4A) Illustration for preparation of drug-loaded RBCs. To test whether protein drugs can be encapsulated efficiently by electroporation, Alexa 488-labeled transferrin, an iron-binding blood plasma glycoprotein, was loaded to RBCs by electroporation (Nucleofector 2b from Lonza) using program A-023. 100 μg Alexa 488-labeled transferrin proteins (Jackson Immunochemical) mixed with 100 million RBCs were subjected to electroporation for encapsulation in 1 ml IDMD media. (FIG. 4B) After electroporation, cells were washed with 1 g/L glucose-containing PBS, followed by flow cytometry to measure electroporation efficiency. Alexa 488-positive cells were considered as transferrin-loaded RBCs.

FIGS. 5A to 5B: Electroporation programs were tested to optimize RBC viability and encapsulation efficiency. (FIG. 5A) Transferrin was subjected to electroporation (Nucleofector 2b from Lonza) with indicated programs for optimal encapsulation of protein drugs as described in FIG. 4A. RBC viability (%) indicates the frequency of recovered RBC numbers relative to initial RBC numbers. (FIG. 5B) Electroporation efficiencies with different electroporation programs were analyzed by flow cytometry as described in FIG. 4B. Alexa 488-positive cells were considered as transferrin-loaded RBCs, showing A-023 as an optimal electroporation program.

FIGS. 6A to 6B: Optimization of electroporation medium for RBC viability and encapsulation efficiency. (FIG. 6A) Transferrin was subjected to electroporation (Nucleofector 2b from Lonza) with indicated medium for optimal encapsulation of protein drugs as described in FIG. 4A. Bar graph shows that RBC viability (%) is highest with IMDM. (FIG. 6B) Electroporation efficiencies with different electroporation medium were analyzed by flow cytometry as described in FIG. 4B, showing IMDM as an optimal electroporation medium.

FIG. 7A to 7B: Optimization of RBC and protein concentrations for encapsulation efficiency. (FIG. 7A) Transferrin was subjected to electroporation (Nucleofector 2b from Lonza) with indicated cell and protein concentration for optimal encapsulation of protein drugs as described in FIG. 4A, showing 10 million RBCs/100 ul and 200 ug proteins/ml as optimal RBC and protein concentrations, respectively. (FIG. 7B) RBCs without electroporation were used as a control.

FIG. 8A to 8B: Transfused RBCs deliver encapsulated FITC-labeled CD31 antibodies (Ab) to the sites of RBC aggregates and label endothelial cells adjacent to RBC aggregates in SCD mouse liver. RBCs (300 million) were electroporated to encapsulate 100 μg of FITC-labelled CD31 antibodies and transfused to SCD mice, followed by TNFα-injection for 1 h. From PBS-perfused mice transcardially 1 h after TNFα injection, the organs (bone marrow, spleen and liver) were harvested and fixed by PLP fixative, followed by cryosectioning. Frozen liver sections were stained with Ter119 antibody for RBCs, anti-endoglin antibody for sinusoids, and anti-FITC antibody (Mouse IgG monoclonal; Jackson Immunochemical) to label RBC encapsulated antibodies, and DAPI for nuclei. Alexa488-labeled anti-FITC antibody was used to amplify CD31 antibody signals. (FIG. 8A) A representative 2D LaSC image with Endoglin and CD31 signals is shown in the left panel, while a merged image with DAPI and Ter119 signals is shown in the right panel. Arrows indicate that CD31-labeled intravascular endothelial cells next to RBC aggregates. (FIG. 8B) A representative confocal image was processed using Imaris software (Bitplane, Zurich, Switzerland) to render 3D reconstructed images in the liver. Asterisks indicate CD31-labeled intravascular endothelial cells next to RBC aggregates.

FIG. 9: Transfused RBCs provide targeted delivery of encapsulated FITC-CD31 antibodies to the sites of RBC aggregates in SCD mouse liver. The # of CD31-labeled endothelial cells (ECs) per 1000 hepatocytes were quantitated by 2D laser scanning cytometry (LaSC; iCys by CompuCyte) from FIG. 8A. Systemic i.v. injection of FITC-labelled CD31 antibody was used as a control. Student t-test, p<0.05 (n=3 mice).

FIG. 10: The in vivo half-life of P-selectin antibody-loaded RBCs is similar to untreated healthy RBCs in SCD mice. Fresh RBCs were electroporated to encapsulate P-selectin antibodies. The RBC-encapsulated P-selectin antibody (P-sel-RBC) and fresh RBCs were labeled with CFSE and Deep Red (Invitrogen) dyes, respectively, followed by i.v. injection to SCD mice (40 million each per mouse). Blood cells from injected mouse tails were collected at indicated time for 4 weeks and subjected to flow cytometry analysis. The frequency of circulating CFSE-labeled P-sel-RBCs and Deep Red-labeled fresh RBCs were calculated by flow cytometry. The frequencies of transfused RBC survival were quantitated by the transfused RBC survival(%) =frequency of transfused RBCs on indicated time/frequency of transfused RBCs at injection×100. Student t-test between P-sel-RBCs and fresh RBCs did not show significant difference (n=6 mice).

FIG. 11: The in vitro half-life of P-selectin Ab-loaded RBCs is comparable to fresh RBCs. Fresh RBCs were electroporated to encapsulate P-selectin antibodies. IMDM medium alone was used as an electroporation control. The RBC-encapsulated P-selectin antibodies (P-sel-RBC), electroporated RBC control, and fresh RBCs were resuspended in citrate-phosphate-dextrose solution (sigma) and stored in 96-well plates at 4° C. Stored cells were subjected to flow cytometry at indicated time points. The concentrations of surviving P-sel-RBCs, electroporated RBC control, and fresh RBCs were calculated from the flow cytometry. The frequencies of in vitro RBC survival were quantitated by the RBC survival (%)=concentration of RBCs on indicated time/concentration of RBCs at the beginning of storage×100. Student t-test between P-sel-RBCs and fresh RBCs did not show significant difference (n=6 mice).

FIG. 12: Parameters of RBC aggregates were determined to quantitate vaso-occlusion in SCD mouse liver. SCD mice were injected with TNFα (1 μg/mouse; 1 h) to induce inflammatory vaso-occlusion, followed by CO₂ euthanasia and PBS perfusion to flush unbound RBCs in the vasculature. The livers were harvested and fixed by PLP fixative, followed by cryosectioning and immunofluorescent staining with antibodies against Ter119 (for RBC marker), Endoglin (for sinusoidal vasculature), S100A8 (for neutrophils), and DAPI (for nuclei). Stained slides with fluorescent dye-labeled secondary antibodies were scanned on laser scan cytometry (LaSC), followed by quantitative imaging analysis (iCys software) to quantitate the # of RBC aggregates that are larger than 100 μm², total area of RBC aggregates, the # neutrophil-containing RBC aggregates by iCys software. Arrows indicate the neutrophil-containing RBC aggregate in liver sinusoid.

FIG. 13: Representative LaSC images of liver cryosections show that RBC aggregates are increased in SCD SS mice with a further increase upon TNFα injection. SCD SS and control AA mice were injected with or without TNFα (1 μg/mouse; 1 h) to induce inflammatory vaso-occlusion, followed by CO₂ euthanasia and PBS perfusion to flush unbound RBCs in the vasculature. Immunofluorescent staining of liver cryosections was performed as described in FIG. 12 to produce LaSC images, which were subjected to quantitative imaging analysis of RBC aggregates for 3 parameters in SCD mouse livers (see FIG. 14).

FIG. 14: LaSC analysis strategy shows automatic image segmentation to generate contour-based events. From FIG. 13 LaSC scanning, cytometric events are defined by automatic image segmentation on specific channels by iCys software to quantitate # of RBC aggregate, total area of RBC aggregates, # of neutrophil-containing RBC aggregates by iCys software (Park et al. 2020). Arrows indicate that the images of individual channels were processed with events-generating contours, followed by the generation of representative images and graphs in FIG. 15.

FIGS. 15A to 15B: Quantitative measurement of RBC aggregates shows 3 parameters in SCD mouse liver after TNFα injection from the LaSC analysis in FIG. 14. (FIG. 15A) Representative images show the increased RBC aggregates upon TNFα injection in SCD mouse liver. (FIG. 15B) This was followed by quantitative imaging analysis (iCys software) to quantitate # of RBC aggregate, total area of RBC aggregates, # of neutrophil-containing RBC aggregates by iCys software. Bar graphs are plotted as mean±SEM, n=4 mice per group. Student's t-test, **p<0.01.

FIGS. 16A to 16B: RBC-encapsulated P-selectin Ab reduces RBC-aggregates during steady state conditions in SCD mouse liver. (FIG. 16A) SCD mice were generated by BM reconstitution of C57BL/6 wild type mice for 4 months from SS donor BM cells (SS→WT chimera mice). Control chimera mice were generated from AA donor BM cells (AA→WT chimera mice). A schematic shows that SCD or control mice were injected with drug-encapsulated RBC by i.v. at t=0, as indicated in the left arrow. After 3 days, the mice were sacrificed by CO₂ euthanasia, followed by PBS perfusion to flush unbound RBCs in the vasculature. The livers were harvested and fixed by PLP fixative, followed by cryosectioning and immunofluorescent staining with antibodies against Ter119 (for RBC marker), Endoglin (for sinusoidal vasculature), S100A8 (for neutrophils), and DAPI (for nucleus). (FIG. 16B) Stained frozen section slides were scanned on LaSC, followed by quantitative imaging analysis (iCys software) to show # of RBC aggregates, total area of RBC aggregates, and # of neutrophil-containing RBC aggregates. Bar graphs are plotted as mean+SEM, n=4 mice per group. Student's t-test, *p<0.05, **p<0.01.

FIG. 17: RBC-encapsulated P-selectin Ab reduces the numbers of RBC-aggregates in small size (50 -100 μm²) as well as large size (>100 μm²) from SCD mouse liver, indicating that the effect of RBC-encapsulated P-selectin Ab is not limited to large RBC aggregates. SCD mice (SS→WT chimeras) were injected with P-selectin Ab-encapsulated RBC or control RBC (i.v.). After 3 days, the mice were sacrificed by CO₂ euthanasia, followed by PBS perfusion to flush unbound RBCs in the vasculature. The livers were harvested and processed as described in FIG. 16A, followed by quantitative imaging analysis (iCys software) to quantitate # of RBC aggregates vs. the size of RBC aggregates in three different groups (10-50 μm²; 50-100 μm²; >100 μm²) by iCys software. Bar graphs are plotted as mean+SEM, n=4 mice per group. Student's t-test, *p<0.05, **p<0.01.

FIG. 18: RBC-encapsulated P-selectin Ab reduces the numbers of neutrophil-containing RBC aggregates in all different sizes from SCD mouse liver. From SCD mice (SS→WT chimeras) injected with P-selectin Ab-encapsulated RBC or control RBC (i.v.), 3 days as described in FIG. 17, the numbers of neutrophil-containing RBC aggregates in three different groups of RBC aggregates were quantitated by iCys software. Bar graphs are plotted as mean+SEM, n=4 mice per group. Student's t-test, *p<0.05, **p<0.01.

FIGS. 19A to 19B: RBC-encapsulated P-selectin Ab reduces RBC-aggregates 24 h after treatment in SCD mouse liver upon TNFα injection. (FIG. 19A) A schematic shows that SCD or control mice were injected with indicated drug-encapsulated RBC (i.v.). After 24 hours, the mice were subjected to TNFα injection (i.p. for 1 h) before CO₂ euthanasia, followed by PBS perfusion to flush unbound RBCs in the vasculature. Livers were harvested and processed as described in FIG. 16A. (FIG. 19B) Stained frozen section slides were scanned on LaSC, followed by quantitative imaging analysis (iCys software). Bar graphs are plotted as mean+SEM, n=4 mice per group. Student's t-test, *p<0.05, **p<0.01.

FIGS. 20A to 20B: RBC-encapsulated P-selectin Ab reduces RBC-aggregates 3 days after treatment in SCD mouse liver upon TNFα injection. (FIG. 20A) A schematic shows that SCD or control mice were injected with indicated drug-encapsulated RBC (i.v.) for 3 days before TNFα injection, followed by procedures described in FIG. 19A. (FIG. 20B) Stained slides with fluorescent dye-labeled secondary antibodies were scanned on LaSC, followed by quantitative imaging analysis (iCys software). Bar graphs are plotted as mean+SEM, n=4 mice per group. Student's t-test, *p<0.05, **p<0.01.

FIGS. 21A to 21B: RBC-encapsulated Alteplase reduces RBC aggregates during steady state conditions in SCD mouse liver. (FIG. 21A) A schematic shows that SCD or control mice were injected with indicated drug-encapsulated RBC (i.v.) for 3 days, followed by CO₂ euthanasia and subsequent procedures described in FIG. 19A. (FIG. 21B) Stained slides with fluorescent dye-labeled secondary antibodies were scanned on LaSC, followed by quantitative imaging analysis (iCys software). Bar graphs are plotted as mean+SEM, n=4 mice per group. Student's t-test, *p<0.05, **p<0.01.

FIGS. 22A to 22B: RBC-encapsulated Alteplase reduces RBC-aggregates 24 h after treatment in SCD mouse liver upon TNFα-injection. (FIG. 22A) A schematic shows that SCD or control mice were injected with indicated drug-encapsulated RBC (i.v.) for 24 h before TNFα treatment (1 h), followed by CO₂ euthanasia and subsequent procedures described in FIG. 19A. (FIG. 22B) Stained slides with fluorescent dye-labeled secondary antibodies were scanned on LaSC, followed by quantitative imaging analysis (iCys software). Bar graphs are plotted as mean+SEM, n=4 mice per group. Student's t-test, *p<0.05, **p<0.01.

FIGS. 23A to 23B: RBC-encapsulated Alteplase reduces RBC-aggregates 3 days after treatment in SCD mouse liver upon TNFα-injection. (FIG. 23A) A schematic shows that SCD or control mice were injected with indicated drug-encapsulated RBC (i.v.) for 3 days before TNFα treatment (1 h), followed by CO₂ euthanasia and subsequent procedures described in FIG. 19A. (FIG. 23B) Stained slides with fluorescent dye-labeled secondary antibodies were scanned on LaSC, followed by quantitative imaging analysis (iCys software). Bar graphs are plotted as mean+SEM, n=4 mice per group. Student's t-test, *p<0.05, **p<0.01.

FIG. 24: RBC-encapsulated P-selectin Ab and Alteplase reduce the sizes of enlarged spleens 3 days after treatment in SCD mice. A schematic shows that SCD or control mice were injected with indicated drug-encapsulated RBC (i.v.) for 3 days, followed by CO₂ euthanasia and PBS perfusion to flush unbound RBCs in the vasculature. Spleens were harvested to measure the weight. Bar graphs are plotted as mean+SEM, n=4 mice per group. Student's t-test, *p<0.05, **p<0.01.

FIG. 25: RBC-encapsulated Alteplase restores B cell follicles 3 days after treatment in the spleen of SCD mice. SCD or control mice were injected with indicated drug-encapsulated RBC (i.v.) for 3 days, followed by CO₂ euthanasia and PBS perfusion. Spleens were harvested and fixed with PLP fixative, followed by cryosectioning and immunofluorescent staining with antibodies against Ter119 (for RBC marker), Endoglin (for sinusoidal vasculature), B220 (for B cells), and DAPI (for nucleus). Stained slides with fluorescent dye-labeled secondary antibodies were scanned on LaSC. Representative images are shown from 4 experiments.

FIGS. 26A to 26C: RBC-encapsulated P-selectin antibody and Alteplase reduce vascular RBC aggregates 3 days after treatment in the kidneys of SCD mice upon TNFα injection. (FIG. 26A) A schematic shows that SCD or control mice were injected with indicated drug-encapsulated RBC (i.v.) for 3 days before TNFα injection (1 h), followed by CO₂ euthanasia and subsequent procedures for fluorescent staining of kidneys as described in FIG. 19A. (FIG. 26B) Stained slides were scanned on LaSC, followed by quantitative imaging analysis (iCys software) to show # of RBC aggregates per 1 mm² in kidneys. Bar graphs are plotted as mean+SEM, n=4 mice per group. Student's t-test, *p<0.05, **p<0.01. (FIG. 26C) Stained slides were scanned on LaSC, followed by quantitative imaging analysis (iCys software) to quantitate the % area of RBC aggregates in kidney. Bar graphs are plotted as mean+SEM, n=4 mice per group. Student's t-test, *p<0.05, **p<0.01.

FIG. 27: Representative LaSC images show that RBC-encapsulated P-selectin Ab and Alteplase reduce renal vascular congestion 3 days after treatment in SCD mice upon TNFα injection. From the experiments described in 26A, LaSC images were acquired from kidney medulla sections stained with fluorescence dye-labeled Ter119, S100A8, and endoglin antibodies. TNFα-injected SS mice show vascular congestion, i.e. the aggregation of RBCs in the capillary of the renal medulla. The vascular congestion was reduced by the treatment of RBC-encapsulated P-selectin Ab and Alteplase for 3 days. Yellow contours indicate Ter119+ RBC aggregates (>100 μm²).

FIG. 28: A schematic shows how to analyze the effect of RBC -encapsulated drug treatment on RBC aggregates during steady state conditions in humanized SCD mice. SCD or control mice were injected with indicated drug-encapsulated RBC (i.v.) for 3 days, followed by CO₂ euthanasia and subsequent procedures for fluorescent staining of brains and hearts as described in FIG. 19A.

FIGS. 29A to 29B: SCD mouse brain analysis shows that RBC-encapsulated P-selectin Ab and Alteplase reduce RBC aggregates. SCD or control mice were injected with indicated drug-encapsulated RBC (i.v.) for 3 days and processed as described in FIG. 28. (FIG. 29A) From quantitative LaSC analysis (iCys software) of brain sections, bar graph shows # of RBC aggregate per mm² area in brain sections. Bar graphs are plotted as mean+SEM, n=4 mice per group. Student's t-test, *p<0.05, **p<0.01. (FIG. 29B) From quantitative LaSC analysis (iCys software) of brain sections, bar graph shows % area of RBC aggregates in the scanned area. Bar graphs are plotted as mean+SEM, n=4 mice per group. Student's t-test, *p<0.05, **p<0.01.

FIGS. 30A to 30B: SCD mouse heart analysis shows that RBC-encapsulated P-selectin Ab and Alteplase reduce RBC aggregates. SCD or control mice were injected with indicated drug-encapsulated RBC (i.v.) for 3 days and processed as described in FIG. 28. (FIG. 30A) From quantitative LaSC analysis (iCys software) of heart sections, bar graph shows # of RBC aggregate per mm² area in heart sections. Bar graphs are plotted as mean+SEM, n=4 mice per group. Student's t-test, *p<0.05, **p<0.01. (FIG. 30B) From quantitative LaSC analysis (iCys software) of heart sections, bar graph shows % area of RBC aggregates in the scanned area. Bar graphs are plotted as mean+SEM, n=4 mice per group. Student's t-test, *p<0.05, **p<0.01.

FIG. 31: A schematic shows how to analyze the effect of prophylactic RBC-encapsulated drug treatment for 3 days on TNFα-induced vascular congestion with RBC aggregates in humanized SCD mice. A schematic shows that SCD or control mice were injected with indicated drug-encapsulated RBC (i.v.) for 3 days, followed by TNFα injection (1 h) and subsequent procedures for fluorescent staining of brains and hearts as described in FIG. 19A.

FIGS. 32A to 32B: SCD mouse brain analysis shows that RBC-encapsulated Alteplase reduce RBC aggregates upon TNFα injection. SCD or control mice were injected with indicated drug-encapsulated RBC (i.v.) for 3 days before TNFα injection (1 h), followed by procedures as described in FIG. 31. (FIG. 32A) From quantitative LaSC analysis (iCys software) of brain sections, bar graph shows # of RBC aggregate per mm² area. Bar graphs are plotted as mean+SEM, n=4 mice per group. Student's t-test, *p<0.05, **p<0.01. (FIG. 32B) From quantitative LaSC analysis (iCys software) of brain sections, bar graph shows % area of RBC aggregates in the scanned area. Bar graphs are plotted as mean+SEM, n=4 mice per group. Student's t-test, *p<0.05, **p<0.01.

FIG. 33: Representative images show that RBC-encapsulated P-selectin Ab and Alteplase reduce brain vascular congestion 3 days after treatment in SCD mice upon TNFα injection. As described in FIG. 31, LaSC images were acquired from brain sections stained with fluorescence dye-labeled Ter119, S100A8, and endoglin antibodies and DAPI. Zoomed images show a large RBC aggregate from SS (RBC+TNFα) and small RBC aggregates from AA, SS (P-selectin Ab-RBC+TNFα), or SS (Alteplase-RBC+TNFα) in high magnification. TNFα-injected SS mice show vascular congestion with RBC aggregates in the capillary of the brain. The vascular congestion was reduced by the treatment of RBC-encapsulated P-selectin Ab and Alteplase for 3 days. Yellow contours indicate Ter119+ RBC aggregates (>100 μm²).

FIGS. 34A to 34B: SCD mouse heart analysis shows that RBC-encapsulated Alteplase reduce RBC aggregates upon TNFα injection. SCD or control mice were injected with indicated drug-encapsulated RBC (i.v.) for 3 days before TNFα injection (1 h), followed by procedures as described in FIG. 31. (FIG. 34A) From quantitative LaSC analysis (iCys software) of heart sections, bar graph shows # of RBC aggregate per mm² area. Bar graphs are plotted as mean+SEM, n=4 mice per group. Student's t-test, *p<0.05, **p<0.01. (FIG. 34B) From quantitative LaSC analysis (iCys software) of heart sections, bar graph shows % area of RBC aggregates in the scanned area. Bar graphs are plotted as mean+SEM, n=4 mice per group. Student's t-test, *p<0.05, **p<0.01.

FIG. 35: Representative images show that RBC-encapsulated Alteplase reduce heart vascular congestion 3 days after treatment in SCD mice upon TNFα injection. As described in FIG. 31, LaSC images were acquired from heart sections stained with fluorescence dye-labeled Ter119, S100A8, and endoglin antibodies and DAPI. Zoomed images show vessels congested with RBC aggregates from SS (RBC+TNFα) and SS (P-sel Ab-RBC+TNFα) mice, but not from AA (TNFα) and SS (Alteplase-RBC+TNFα) mice in high magnification. The numbers of Ter119+contoured RBC aggregates were significantly reduced in TNFα-injected SCD mice treated with Alteplase-RBC for 3 days compared to SCD mice treated with control RBC. Yellow contours indicate Ter119+ RBC aggregates (>100 μm²).

FIGS. 36A to 36B: TNFα induces widespread intra-sinusoidal fibrin clot formation in SCD mouse bone marrow, arrows indicate fibrin clots. (FIG. 36A) SCD mice were subjected to TNFα injection (i.p. for 1 h) before CO₂ euthanasia, followed by PBS perfusion to flush unbound RBCs in the vasculature. Femurs were harvested and fixed by PLP fixative, followed by cryosectioning and immunofluorescent staining with antibodies against Ter119 (for RBC marker), Endoglin (for sinusoidal vasculature), Fibrinogen/fibrin (for thrombotic clot), and DAPI (for nucleus). Stained slides with fluorescent dye-labeled secondary antibodies were scanned on laser scan cytometry (LaSC). A representative image is shown from 4 independent experiments with similar result. (FIG. 36B) From rectangle area of FIG. 36A, a magnified image is shown. The blue arrows indicate some of fibrin clots in RBC aggregates from SCD mouse BM sinusoids, suggesting that i) fibrin may mediate the hemolysis of transfused drug-loaded RBCs to deliver drugs in situ at the sites of RBC aggregates; ii) fibrin clots can also be targets for RBC-encapsulated thrombolytic drug.

FIG. 37: An illustration shows that in situ targeted drug delivery to the sites of intravascular heterotypic cell aggregates reduces vaso-occlusion in SCD. Step 1. Transfused drug-containing RBCs flow in vessels. Step 2. Transfused drug-containing RBCs are recruited by RBCs aggregates bound to activated endothelial cells, platelets, and neutrophils. Step 3. Transfused drug-containing RBCs are hemolyzed and unload the encapsulated drug to the site of RBC aggregates. Step 4. The targeted drug reduces RBC aggregates/vaso-occlusion.

### DETAILED DESCRIPTION OF CERTAIN EMBODIMENTS

Currently, there are limitations for developing new drugs targeting complex pathophysiological processes (e.g., vasoocclusion) in sickle cell disease (SCD). The systemic injection of those drugs has limited effectiveness due to toxicity from high doses and/or chronic use (e.g., fibrinolysis and unwanted bleeding), rapid clearance, and immunogenic risk. The present disclosure, in some aspects, provides a composition comprising a red blood cell (RBC) and a vasoocclusion-inhibiting agent wherein the vasoocclusion-inhibiting agent is encapsulated in the RBC. A vasoocclusion-inhibiting agent encapsulated in a RBC is a promising therapeutic alternative for the administration of toxic or rapidly cleared drugs. The in situ delivery of a vasoocclusion-inhibiting agent encapsulated in red blood cells represents a novel and unexplored drug delivery to treat vasoocclusion in SCD. Accordingly, some aspects of the present disclosure provide a composition comprising a red blood cell (RBC) and a vasoocclusion-inhibiting agent wherein the vasoocclusion-inhibiting agent is encapsulated in the RBC.

A “red blood cell” is a blood cell that delivers oxygen to tissue or a cell in the body via blood flow through the circulatory system. In humans, mature red blood cells are flexible oval biconcave disks. There are 20-30 trillion red blood cells in the human body and nearly half of the blood's volume is red blood cells. In some embodiments of the present disclosure, the RBC is an autologous RBC. In some embodiments, the RBC is an allogenic RBC.

A “vasoocclusion-inhibiting agent” refers to an agent that inhibits the formation of a vasoocclusion or promotes the resolution of a vasoocclusion. Vasoocclusion is the obstruction of circulation in a blood vessel. In some embodiments, the blood vessel occlusion is caused by sickle cell disease (SCD). SCD is a disorder that affects hemoglobin, which is the molecule in the RBC that carries and delivers oxygen to cells and tissues in the body. Subjects with SCD have atypical hemoglobin molecules which can distort red blood cells into a sickle or crescent shape. SCD may lead to hemolysis, inflammation, activation of blood cells and endothelial cells, and multicellular adhesion in vasculature, resulting in vasoocclusion, vasoocclusive crisis (VOC), that can eventually lead to end-organ damage.

Blood vessel occlusion is a blockage of a blood vessel (e.g., artery or vein) with, for example a clot, or a RBC aggregate, or a heterocellular aggregate, which causes cells, tissues, and organs to become deprived of oxygen. The occlusion can be partial or complete. Blood vessel occlusion is associated with severe pain, stroke, acute chest syndrome, pulmonary hypertension, and other chronic morbidities. It can lead to organ damage, especially in the lungs, kidneys, spleen, liver, heart, and brain. Blood vessel occlusion occurs in a variety of diseases and disorders, such as, for example, sickle cell disease (SCD), peripheral arterial disease, May-Thurner syndrome, carotid artery disease, and venous and arterial thrombosis.

Provided herein are compositions comprising a vasoocclusion-inhibiting agent encapsulated in a RBC. The terms “vasoocclusion-inhibiting agents encapsulated in a RBC” is interchangeable with the term “RBC encapsulated vasoocclusion-inhibiting agent”. Several vasoocclusion-inhibiting agents are candidates for RBC encapsulation in the context of the instant disclosure. These include anti-adhesion agents, tissue plasminogen activators, anti-coagulants, anti-platelet agents, endothelin antagonists, anti-inflammatory agents, agents that counteract free hemoglobin, heme, or iron, and modulators of ischaemia-reperfusion and oxidative stress (Telen et al. 2019).

Anti-adhesion agents (for SCD, for example), include agents which interfere with selectin-mediated adhesion. Selectins are expressed by endothelial cells, platelets, and leukocytes, and selectin receptors are present on all blood cells. In general, selectins mediate cell to cell interactions and therefore facilitate rolling, a phenomenon in which cells have intermittent or continuous contact with endothelial cells as they circulate through the blood vessel. These interactions lead to firmer attachment through integrin-mediated interactions, which result in a stable attachment to the endothelial layer. Leukocytes with selectins can migrate through the endothelial layer into tissues in the presence of appropriate chemokine signals. Examples of selectins that are important to the pathophysiological mechanism of vasoocclusion include E-selectins and P-selectins. Non-limiting, exemplary anti-adhesion agents that may be used in the present disclosure include: Rivipansel (Telen et al. 2015), Crizanlizumab (Anti-P-selectin antibody) (Ataga et al. 2016), Sevuparin (Telen et al. 2016), Nonsulfated heparins (Alshaiban et al. 2016), Anti-selectin inhibitors (Burnette et al. 2011, Gutsaeva et al. 2011), Phosphodiesterase 9 inhibitors (BAY73-6691, PF-04447943, IMR-687) (McArthur et al. 2016).

In some embodiments, the anti-adhesion agent is an anti-P-selectin agent. P-selectin functions as a cell adhesion molecule on the surface of activated endothelial cells, which line the inner surface of blood vessels (i.e., endothelial cells) and on activated platelets. P-selectin can be translocated from secretory granules in endothelial cells and platelets by pro-thrombotic and inflammatory factors generated upon cell activation, and can be localized to the surface of the cells. The surface expressed P-selectin can promote accumulation of platelets and leukocytes that will promote vein wall injury and thrombus formation. In some embodiments, the anti-P-selectin agent is an anti-P-selectin antibody. The anti-P-selectin antibody can be a polyclonal antibody or a monoclonal antibody. In some embodiments, the anti-P-selectin antibody is Crizanlizumab (Ataga et al. 2016).

In some embodiments, the vasoocclusion-inhibiting agent is a tissue plasminogen activator. A “tissue plasminogen activator” refers to a thrombolytic protease that converts inactive plasminogen into active plasmin, which then degrades fibrin complexes in the thrombus. In some embodiments, the tissue plasminogen activator is Alteplase, Reteplase or Tenecteplase (Collen and Lijnen 2005).

In some embodiments, the vasoocclusion-inhibiting agent is an anti-coagulant. Coagulation activation occurs via multiple mechanisms. For example, in SCD and without wishing to be bound by a specific mechanism, coagulation activation occurs via: (1) Tissue factor (TF)-expressing and phosphatidylserine (PS)-expressing microparticles and abnormal exposure of PS on SRBCs, (2) erythrocyte-platelet and neutrophil-platelet aggregates in the circulation of blood, and (3) endothelial cell activation and hemolysis. Non-limiting, exemplary anticoagulants that may be used in the present disclosure include: Heparin (Chaplin et al. 1989), Low-molecular-mass heparins (Dalteparin) (van Zuuren and Fedorowicz 2015), Rivaroxaban (In phase 2 clinical trial: NCT02072668), apixaban (In phase 3 clinical trial: NCT02179177), Tinzaparin (Qari et al. 2007), and direct thrombin inhibitors (Dabigatrin, Aragatroban, and Lepirudin) (Di Nisio et al. 2005).

In some embodiments, the anti-coagulant is a direct thrombin inhibitor (DTI). DTIs are a class of medications that act as anticoagulants (delaying blood clotting) by directly inhibiting the enzyme thrombin. In some embodiments, the direct thrombin inhibitor is Argatroban, Dabigatrin, or Lepirudin (Di Nisio et al. 2005).

In some embodiments, the vasoocclusion-inhibiting agent is an anti-inflammatory agent. Anti-inflammatory agents are involved in treating issue injury caused by inflammation (such as in SCD, for example). Cellular components including neutrophils, lymphocytes, monocytes, platelets, and pro-inflammatory cytokines are involved in SCD. For example, tumor necrosis factor (TNF) receptor blockers decrease vasoocclusion in mice when given long term. Additionally, antagonist of toll-like receptor 4 (TLR 4) may prevent infusion of heme into SCD mice from causing inflammation, microvascular stasis, and pulmonary injury. Non-limiting, exemplary modulators of anti-inflammatory agents that may be used in the present disclosure include: Regadenoson (Field et al. 2013), Omega-3 fatty acids (Kalish et al. 2015), Etanercept (Solovey et al. 2017), TAK-242 (Belcher et al. 2014), Statins (Hoppe et al. 2017), Dexamethasone (Quinn et al. 2011), AKT2 inhibitor (Barazia et al. 2015), Leukotriene inhibitors (Opene et al. 2014), Endothelin receptor blockade (Koehl et al. 2017), 2-Fluorofucose (Belcher et al. 2015), Complement inhibitors (Schaid et al. 2016), and Curcumin (Valverde et al. 2016).

In some embodiments, the anti-inflammatory agent is an endothelin antagonist. Endothelin 1 (ET1), a potent vasoconstrictor, plays a pro-inflammatory role and endothelin receptor antagonists block inflammatory polymorphonuclear neutrophils and lymphocytes and decrease endothelial cell activation. In some embodiments, the endothelin antagonist is Bosentan (Sabaa et al. 2008, Minniti et al. 2009).

In some embodiments, the vasoocclusion-inhibiting agent is a modulator of ischaemia-reperfusion and oxidative stress. Ischaemia-reperfusion injury caused by the transient vasoocclusion followed by the opening of vessels to reestablish flow (such as what occurs in SCD) leads to increased oxidant production. Reactive oxygen species (ROS) stimulate the production of inflammatory cytokines such as TNF, which upregulates adhesion molecule expression and promotes leukocyte, platelet and procoagulant activation. Non-limiting, exemplary modulators of ischaemia-reperfusion and oxidative stress that may be used in the present disclosure include: N-Acetyl cysteine (Nur et al. 2012), Superoxide dismutase (Wood et al. 2005), Suberoylanilde hydroxamic acid (Vorinostat) (Hebbel et al. 2010), Trimidox (KAUL et al. 2006), Polynitroxyl albumin (Mahaseth et al. 2005), a-Lipoic acid (Martins et al. 2009), L-Carnitine (El-Beshlawy et al. 2006), Glutamine (Niihara et al. 2005), Sirolimus (Jagadeeswaran et al. 2017), and Riociguat (In phase 2 clinical trial: NCT02633397).

In some embodiments, the vasoocclusion-inhibiting agent is an anti-platelet agent. Increased platelet counts and chronic platelet activation is observed in patients with SCD, for example. Chronic platelet activation is caused by increased circulating platelet agonists, such as thrombin, ADP and adrenaline, as well as increased platelet-monocyte and platelet-neutrophil aggregates. Activated platelets promote adhesion of sickle RBCs to the vascular endothelium and contribute to microthrombosis and pulmonary hypertension in SCD patients. Additionally, thrombin and cell-free Hb can trigger platelet activation. Non-limiting, exemplary anti-platelet agents that may be used in the present disclosure include: Prasugrel (Heeney et al. 2016), Ticlopidine (Semple et al. 1984), Piroxicam (Eke et al. 2000), and Eptifibatide (Desai et al. 2013).

In some embodiments, the vasoocclusion-inhibiting agent is an agent that counteracts free hemoglobin, heme, or iron. Hemolysis of sickle RBCs has several negative effects, including decreased NO bioavailability, increased oxidative stress, and arginine depletion (Telen et al. 2019). Non-limiting, exemplary agents that counteract free hemoglobin/heme that may be used in the present disclosure include: Arginine (R-gene 10) (Morris et al. 2013), Tetrahydrobiopterin (Kuvan) (Katusic et al. 2009), Sildenafil (Machado et al. 2011), Haptoglobin (Belcher et al. 2014, Shi et al. 2016), Haemopexin (Vercellotti et al. 2016), and Iron chelators (KAUL et al. 2006, Belcher et al. 2014).

In some embodiments, the vasoocclusion-inhibiting agent is encapsulated in the RBC by ex vivo electroporation (Tsong and Kinosita 1985, Tsong 1991). Encapsulation refers to enclosing an agent inside a RBC without disrupting the plasma membrane integrity.

In some embodiments, the vasoocclusion-inhibiting agent is encapsulated in the RBC by endocytosis (Ginn et al. 1969). In some embodiments, the vasoocclusion-inhibiting agent is encapsulated in the RBC by cell-penetrating peptide (CPP)-mediated internalization (Kwon et al. 2009).

In some embodiments, the vasoocclusion-inhibiting agent is delivered to a specific organ. For example, the reticuloendothelial system (RES) in the spleen and liver (Zocchi et al. 1987).

In some embodiments, the vasoocclusion-inhibiting agent is released at the site of a blood vessel occlusion. Without wishing to be bound by a specific mechanism of action, the RBC encapsulating the vasoocclusion-inhibiting agent may be recruited to the site of occlusion by sickle cell aggregates where the RBC hemolyzes and unloads the encapsulated agent. In some instances, the vasoocclusion-inhibiting agent is released from the RBC by slow diffusion (Foroozesh et al. 2011). The sickle cell aggregates at the site of occlusion may be heterocellular comprising RBCs, white blood cells (WBCs), and platelets.

In some embodiments, the composition further comprises a pharmaceutically acceptable carrier. A “pharmaceutically acceptable carrier” may be a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body.

Each carrier must be “acceptable” in the sense of being compatible with the other ingredients of the formulation and not injurious to the tissue of the patient (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Accordingly, other aspects of the present disclosure provide a method of treating a blood vessel occlusion in a subject (e.g., vasoocclusion in SCD), the method comprising administering to the subject in need thereof an effective amount of the composition described herein.

An “effective amount” is the amount necessary or sufficient to have a desired effect in a subject. The effective amount will vary with the particular condition being treated, the age and physical condition of the subject being treated, the severity of the condition, the duration of the treatment, the nature of the concurrent therapy (if any), the specific route of administration and other factors within the knowledge and expertise of the health care practitioner. This amount will vary from individual to individual and can be determined empirically using known methods by one of ordinary skill in the art. In some embodiments, the effective amount of the composition reduces the size of the occlusion by 10%, 20%, 30%, 40%, 50%, 60%, 70%, 80%, 90%, or 100%.

In some embodiments, the subject is a mammal. In some embodiments, the mammal is a human.

In some embodiments, the composition is administered intravenously. In some embodiments, the composition is administered intravenously at the site or in the vicinity of the site of occlusion. In some embodiments, the composition is administered once. In some embodiments, the composition is administered repeatedly.

In some embodiments, the blood vessel occlusion is caused by sickle cell disease. Blood vessel occlusion can be detected and monitored in sickle cell disease, for example, based on the following parameters: the RBC aggregate or heterocellular aggregate size (e.g., the size of a RBC aggregate or a heterocellular aggregate size is greater than 100 μm² in area), the number of RBC aggregates or heterocellular aggregates, and the extent of neutrophil association with the RBC aggregates or heterocellular aggregates.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1. A Novel Therapeutic Approach To Improve Vaso-Occlusion in Sickle Cell Disease

### Methods

Mouse models and study design: Experiments were performed on 2-8 months-old sex matched healthy control (Hba^{tm1(HAB)Tow} Hbb^{tm3(HBG1,HBB)Tow}; homozygous AA) and humanized Townes SCD (Hba^{tm1(HBA)Tow} Hbb^{tm2(HBG1,HBB*)Tow}; homozygous SS) mice bred in the laboratories of the Boston Children's Hospital (Jackson Laboratory, stock #013071). For the reconstitution of AA or SS mouse BM cells, we used 6-8 week old C57BL/6 wild type (WT) or CXCL12-GFP⁺ reporter mice after 2 split-doses of whole-body irradiation (total 11 Gy) to generate chimera mice with SCD phenotype (SS→WT and SS→CXCL12-GFP⁺ SCD mice) or control AA phenotype (AA→WT and AA→CXCL12-GFP⁺ control chimera mice). The animal protocol was approved by the Boston Children's Hospital Animal Care and Use Committee. See more details in a recent publication (Park et al. 2020).

Mouse PCR genotyping and Flow cytometry analysis of blood cells: Details are reported in a recent publication (Park et al. 2020).

TNFα- and LPS-induced vaso-occlusion: SCD mice were administrated with TNFα (Biolegend; 1 mg per mouse) or LPS (0111:B4 from Sigma; 75 mg per mouse) by intraperitoneal (i.p.) injection under 1.5% isoflurane for 1 h, followed by CO₂ euthanasia and transcardial perfusion PBS.

CFSE labeling and half-life test: RBCs were isolated from fresh WT mouse blood. Blood was collected to 1 g/L glucose-containing PBS, followed by centrifugation with 3 ml Ficoll-Paque™ PLUS (GE Healthcare) at 400 g for 20 min at room temperature. Separated RBCs were transferred to a sterile tube followed by washing twice with glucose-containing PBS. For CFSE (5(6)-Carboxyfluorescein diacetate N-hydroxysuccinimidyl ester) labeling, 200 million RBCs were suspended in PBS without glucose, and mixed with 15 μM CFSE (Invitrogen). Cell mixture was incubated for 12 min at 37° C., followed by washing with glucose-containing PBS. 40 million RBCs were suspended in 100 μl of PBS, and then CFSE-labeled RBCs were administrated to a mouse (100 μl each) by retro-orbital. In vivo half-life was monitored in time-course from tail-tip blood samples by flow cytometry. Transfused RBC survival (%)=frequency of transfused RBCs on indicated time/frequency of transfused RBCs at injection×100.

Drug loading by electroporation: Fresh isolated RBCs were resuspended in IMDM (Invitrogen) to make 100 million/ml final concentration. 100 μg each of anti-P-selectin antibody (RB40.34 clone rat IgG1 from BD Pharmingen or RMP-1 clone mouse IgG2a from Biolegend) or Alteplase (Activase from Genentech) were mixed with 1 ml of RBC suspension. The mixture was transferred to electroporation cuvettes and subjected to electroporation (Nucleofector 2b from Lonza) using program A-023. After washing electroporated RBCs with glucose-containing PBS, 200 μl of drug-loaded RBCs (5 million cells per mouse) were resuspended in PBS and injected to mice by intravenously.

Immunofluorescence staining of cryopreserved sections of organs: Whole mouse femurs and other organs (brain, heart, liver, lung, spleen, kidney) were fixed in phosphate-buffered L-lysine with 1% paraformaldehyde/periodate (PLP) fixative, 16 h at 4° C., followed by washing in 0.1 M phosphate buffer pH 7.5, and cryoprotected for 72 h in 30% sucrose/0.1 M phosphate buffer. The fixed organs were embedded in OCT compound (optimal cutting temperature compound; a water-soluble glycol-resin compound; Sakura Finetek), snap frozen in 2-methylbutane/dry ice mix, and stored at −80° C. Fresh cryopreserved, femurs and other organs were sectioned longitudinally at 5 μm thickness to facilitate in situ laser scanning cytometry (LaSC) analysis of a single cell-thick layer using the CryoJane tape-transfer system (Instrumedics) in a cryostat (LEICA CM1800). All bone sections were prepared from the middle of the femur to include the central sinus. Thawed frozen sections of organs were subjected to immunofluorescent staining with antibodies against endoglin (AF1320; R&D, goat Ab, dilution 1/100), Ter119 (BioLegend, rat Ab, dilution 1/100), and S100A8 (ThermoFisher, rabbit Ab, 1:500). See more details in a recent publication (Park et al. 2020).

In situ solid-phase LaSC analysis: For all LaSC analyses, the iCys Research Imaging Cytometer (CompuCyte) with four excitation lasers (405, 488, 561, and 633 nm), four emission filters (425-455, 500-550, 575-625, 650 nm long pass), and four photomultiplier tubes, was used. For each fluorescent marker, images are built pixel by pixel from the quantitative photomultiplier tube measurements of laser-spot excited fluorescence signals. The quantitative imaging cytometry software iCys generated a single “region” image of organs from a sequence of high-magnification (40×/NA 0.95 dry objective) “field” immunofluorescence images (250 μm×190 μm per field image) that were subjected to automated analysis of contour-based cellular events, their fluorescence levels, and their location within the organ section. Individual cellular events are defined by threshold contouring of DAPI-stained nuclei. Specific types of cellular events, multicellular aggregates, niche components were systemically identified by image segmentation on specific fluorescent signals with iCys analysis software as follows: arterioles/arteries by Sca-1⁺ events, sinusoids by endoglin events, RBC aggregates by TER119⁺ events with area larger than 100 μm², neutrophils by S100A8⁺ cells, hepatocytes by DAPI⁺ events with an area larger than 50 μm² and smaller than 200 μm². Individual cellular and niche events were systematically visualized and confirmed by high resolution images. Isotype antibodies were used as negative controls for gating purposes. The total number and morphological distribution of each distinct cellular subpopulation within the entire organ or at specific anatomical locations within the organ can then be determined using post-scan automated image analysis software (iCys cytometric analysis software; CompuCyte). To assess statistical significance, we analyzed 3 distinct frozen sections per from 4 mice each, total 12 sections. See more details in a recent publication (Park et al. 2020).

Statistical analysis: All data are presented as mean ±SEM (standard error of mean; n =3 to 6). All statistical analysis was performed using Prism 8 software (GraphPad). The significance of difference in the mean values of two conditions was determined using two-tailed Student's t-test unless indicated otherwise. P value less than 0.05 (p<0.05) was considered significant. For graphs representing LaSC data, single femurs from different mice analyzed were considered as independent data points (n=number of mice). When multiple sections from a single femur were analyzed, the results were treated as technical replicates that were averaged and considered as one single independent sample for statistical purposes. Two-tailed unpaired student's t-test *p<0.05, **p<0.01.

### Results

### Transfused RBCs Translocalize to the Vessels of Various Organs in SCD Mice

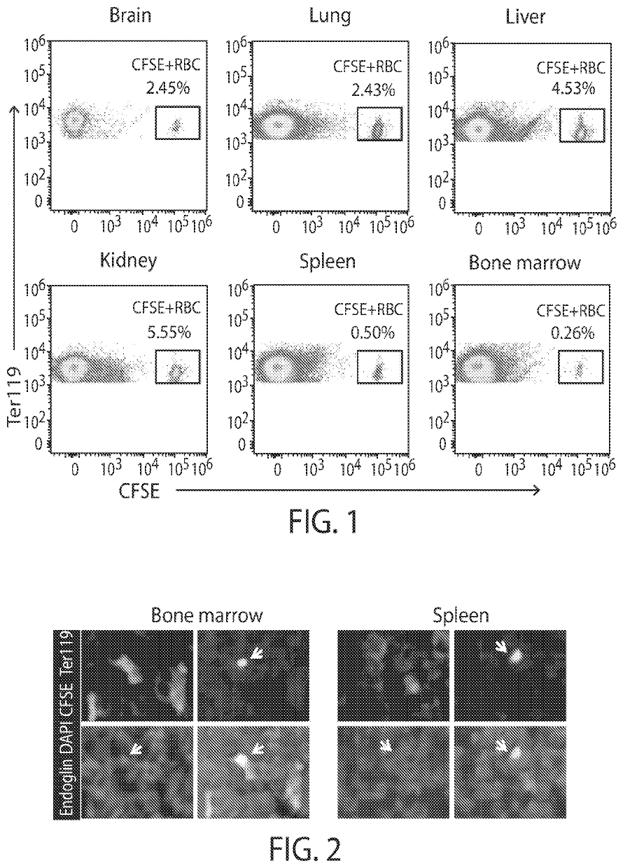

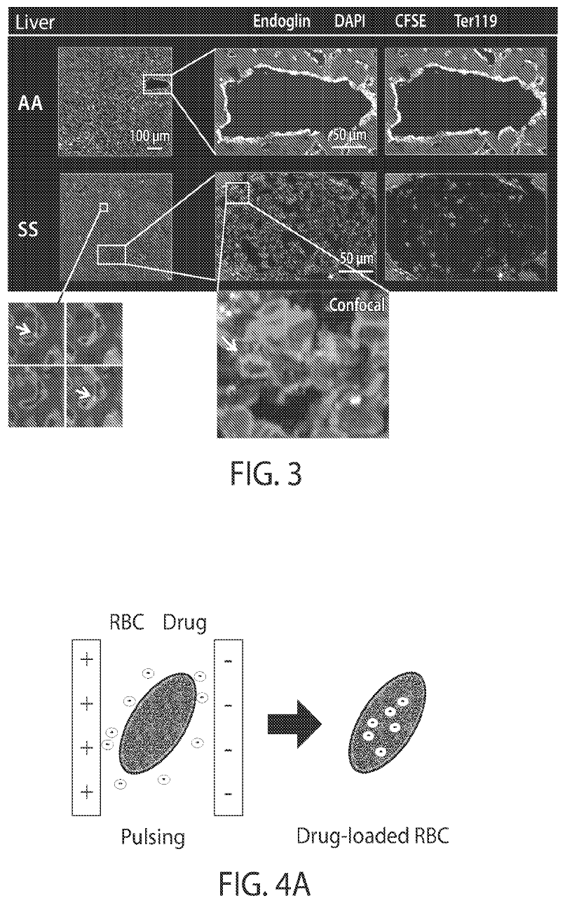

Fresh RBCs from WT mouse were used for CFSE-labeling. One billion RBCs were incubated with 20 uM of CFSE for 12 min at 37° C. (FIGS. 1 and 2). Then CFSE-labeled RBCs were transfused to SCD mice by intravenous injection. First, the localization of CFSE⁺ RBCs were analyzed by flow cytometry in various organs including brain, lung, liver, kidney, spleen and bone marrow 24 h after injection (FIG. 1). The frequencies of CFSE-labeled RBCs show their translocation to all the examined organs upon transfusion. For immunofluorescent imaging analysis, from euthanized mice 24 h after injection, the organs (bone marrow, spleen and liver) were harvested and fixed by PLP, followed by cryosection and immunofluorescent staining with antibodies against Ter119 (for RBC marker), Endoglin (for sinusoidal vasculature), and DAPI (for nucleus) (Park et al. 2020). Laser scanning cytometry (LaSC) analysis (iCys software) shows that the CFSE-labeled transfused RBCs are not only found in the vessels of BM, spleen, and liver, but also incorporated in the RBC aggregates of SCD mouse BM, spleen, and liver (FIGS. 2 and 3). High resolution images from both LaSC and confocal microscopy clearly demonstrate the incorporation of CFSE-labeled transfused RBCs in RBC aggregates of SCD mouse liver (see arrows indicating CFSE-labeled RBCs in RBC aggregates from FIG. 3).

### Protein Drugs Can be Efficiently Encapsulated Into RBCs Ex Vivo by Electroporation

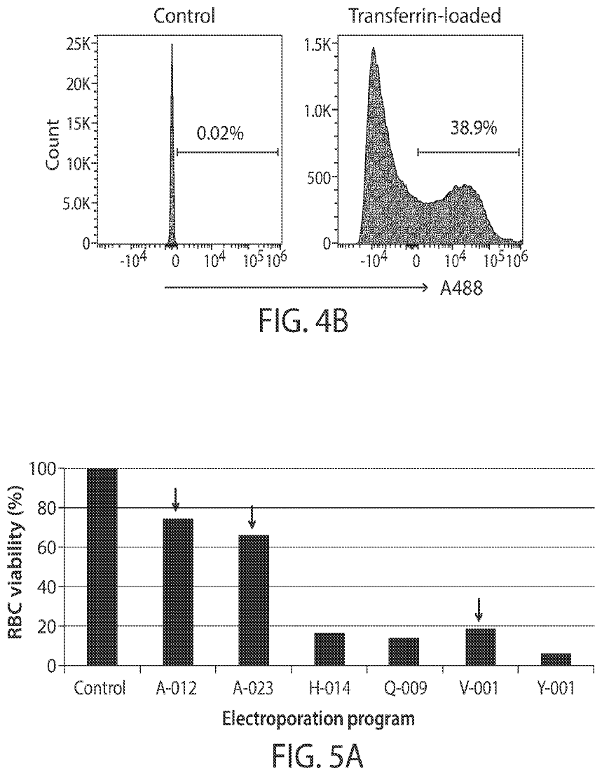

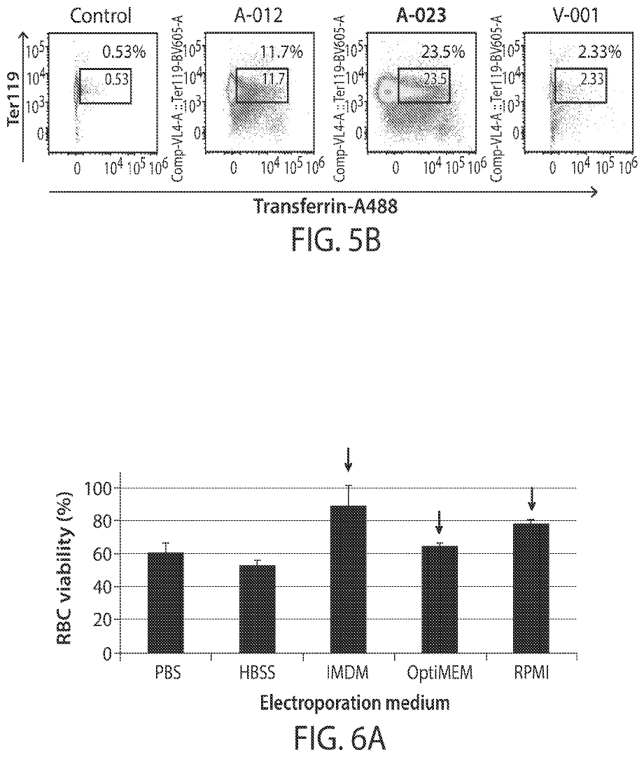

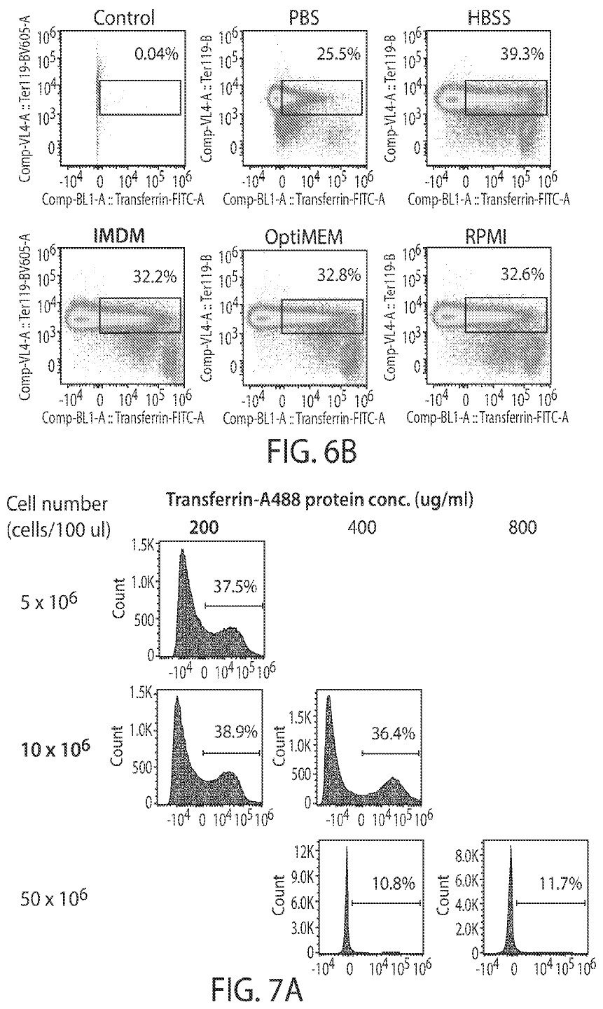

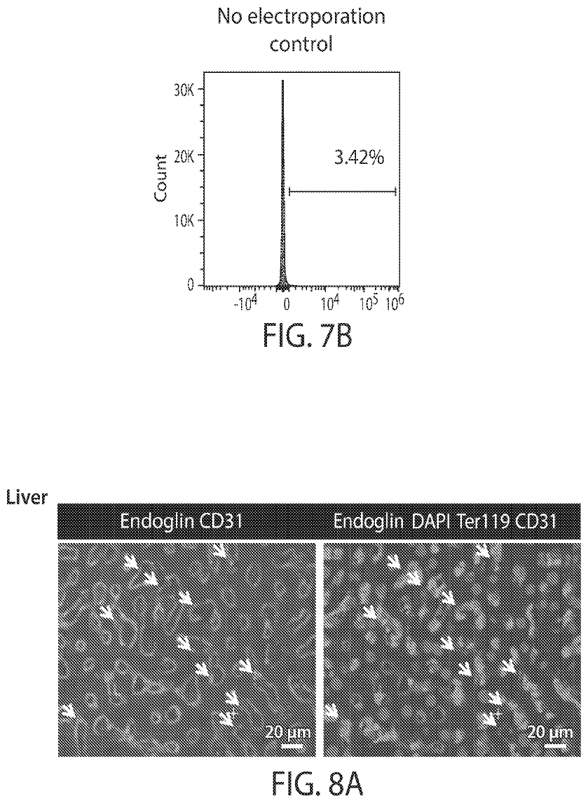

To test the encapsulation of protein drugs into RBCs by electroporation (FIG. 4A), a test protein Alexa 488-labeled transferrin (79 kDa) was encapsulated in RBCs at a cell concentration of 10 million cells/100 ul IMDM medium and a protein concentration of 200 ug/ml (see FIG. 4B) after a series of optimization processes (FIGS. 5A, 5B, 6A, 6B, 7A, and 7B). In optimized condition, RBC viability and encapsulation efficiency were 90% and 39%, respectively (FIGS. 6A and 7A) with healthy hematological parameters (Table 1), indicating electroporation provides efficient encapsulation of proteins into RBCs.

TABLE 1
Transferrin-encapsulated RBCs show
healthy hematological parameters

| Parameter | Control | Loaded |
|---|---|---|
| MCV (mean corpuscular volume; fL) | 45.6 | 45.6 |
| MCH (mean corpuscular Hb; pg) | 14.0 | 14.6 |
| MCHC (mean corpuscular Hb conc.; g/dL) | 30.7 | 32.0 |

### Transfused RBCs Deliver Encapsulated Proteins to the Sites of RBC Aggregates in SCD Mice

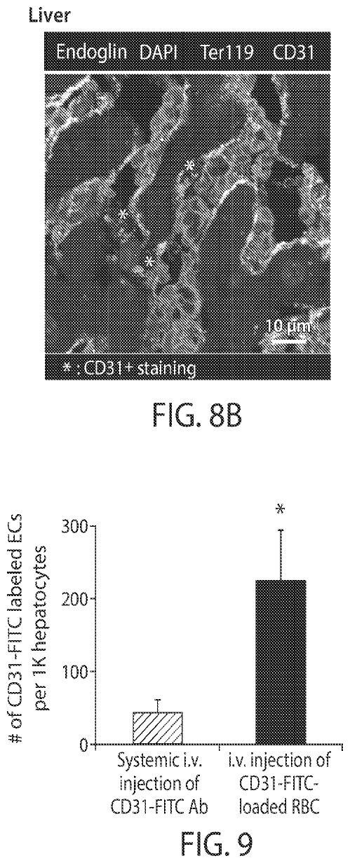

To examine whether transfused RBCs deliver encapsulated proteins to the sites of RBC aggregates in SCD mice, RBCs loaded with FITC-labeled CD31 antibodies were injected intravenously into SCD mice (300 million RBCs/mouse), followed by TNFα injection intraperitoneally to induce vasoocclusion (FIGS. 8 and 9). After 1h, CO₂ euthanized mice were perfused with PBS before harvesting organs for cryosections. The analysis of the liver sections by LaSC and confocal microscopy showed that RBC-encapsulated CD31 antibodies label intrasinusoidal endothelial cells adjacent to RBC aggregates significantly higher than systemically injected FITC-labeled CD31 antibodies (FIGS. 8A, 8B, and 9), suggesting that drug-loaded transfused RBCs can provide in situ targeted drug delivery to sites of intravascular RBC aggregates in SCD.

### The Half-Life of P-Selectin Antibody-Loaded RBCs is Similar to Healthy Untreated RBCs In Vivo in SCD Mice and In Vitro

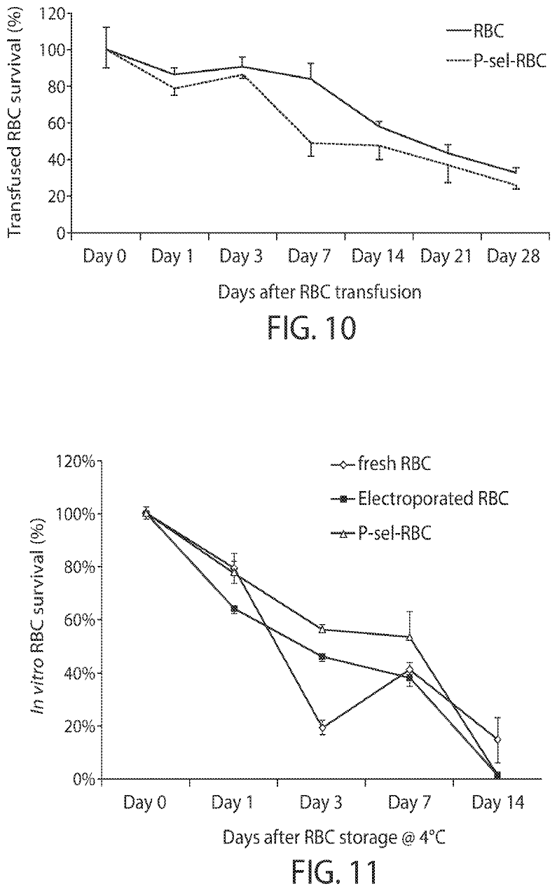

To test whether the in vivo survival of drug-loaded RBCs is compromised after electroporation-mediated drug encapsulation, P-selectin antibody-loaded RBCs and control RBCs, labeled with CFSE and Deep red dyes, respectively, were transfused into SCD mice. The frequencies of circulating CFSE and Deep red labeled RBCs were examined by flow cytometry from tail bleeding on indicated time. The in vivo half-life of P-selectin antibody-loaded RBCs is similar to healthy untreated RBCs in SCD mice (FIG. 10), indicating that the in vivo survival of drug-loaded RBCs is not compromised after electroporation-mediated drug encapsulation.

Next, we tested whether the in vitro survival of drug-loaded RBCs under a storage condition is compromised after electroporation-mediated drug encapsulation. P-selectin Ab-loaded RBCs, electroporated RBCs, and fresh RBCs (10 million/ml) in Citrate-phosphate-dextrose solution (CPD solution used for whole blood storage; Sigma) were stored at 4° C., followed by counting cell numbers at the indicated time (FIG. 11). The in vitro half-life of P-selectin antibody-loaded RBCs is similar to untreated healthy RBCs (FIG. 11), indicating that the in vitro survival of drug-loaded RBCs is not compromised under a storage condition after electroporation-mediated drug encapsulation.

### Three Parameters of RBC Aggregates Were Determined to Quantitate Vaso-Occlusion in SCD Mouse Liver

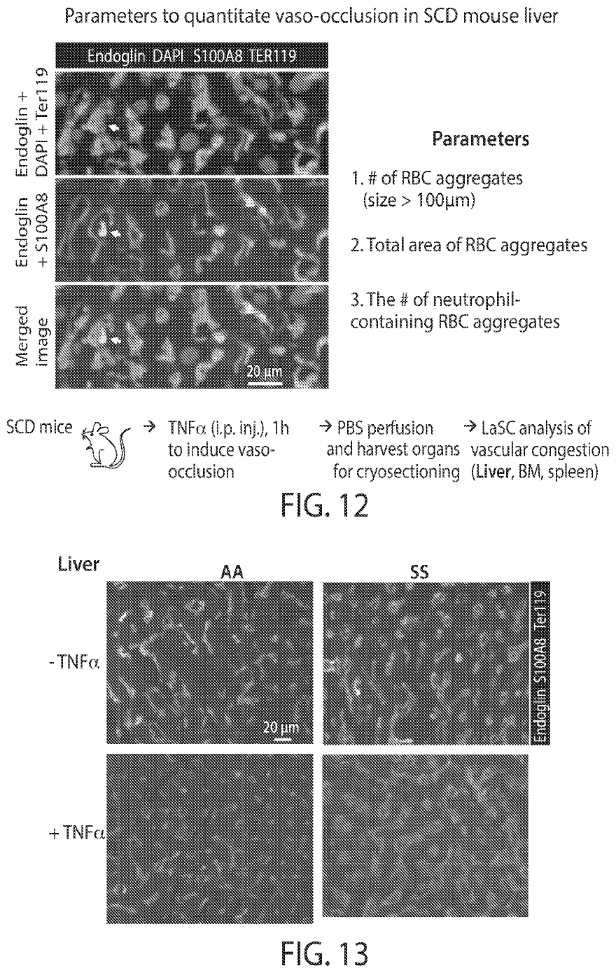

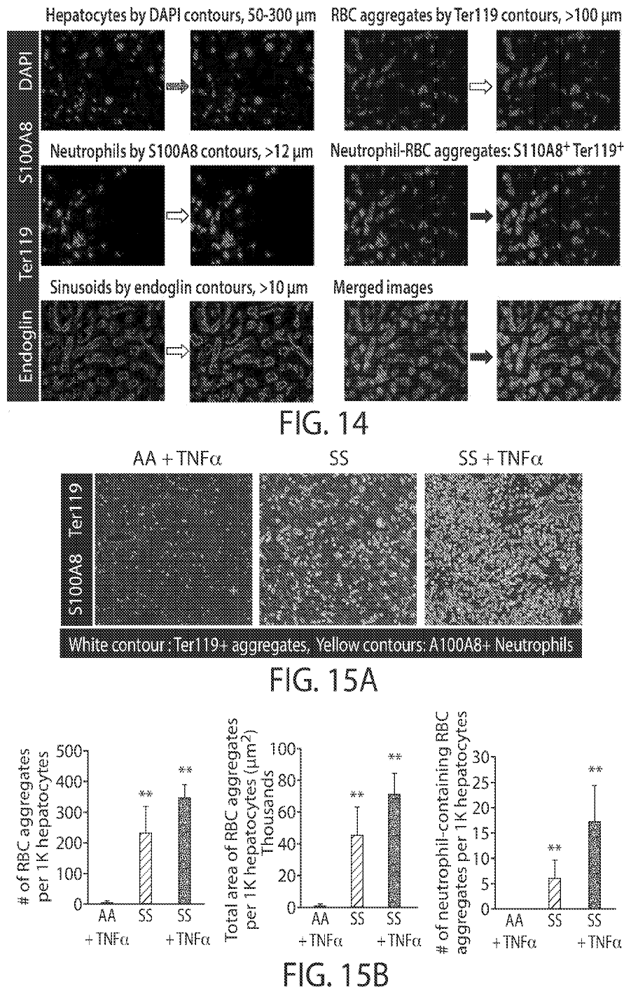

To measure drug effect on vaso-occlusion, we examined parameters of vascular RBC aggregates to quantitate vaso-occlusion in different organs of SCD mice using LaSC. SCD mice were injected with TNFα (1 μg/mouse, 1 h) intraperitoneally to induce vaso-occlusion. After PBS perfusion and harvesting livers for quantitative imaging analysis, we analyzed immunofluorescently stained cryosections by LaSC. From quantitative imaging analysis (iCys software) to quantitate vaso-occlusion in SCD mouse livers, we first determined specific parameters, which include number of RBC aggregates (size >100 μm²), total area of RBC aggregates, and the number of neutrophil-containing RBC aggregates (for association of neutrophil in RBC aggregates) (FIG. 12). Subsequently, LaSC images from immunofluorescently stained liver cryosections of SCD SS mice and control AA mice (FIG. 13) were subjected to the quantitative analysis of RBC aggregates by automatic segmentation on specific channels by iCys software to generate contour-based events (see FIG. 14). The results of the quantitative analysis are shown in FIG. 15A for contour-based events and FIG. 15B for bar graphs of 3 parameters: i) # of RBC aggregates; ii) total area of RBC aggregates; and iii) # of neutrophil-containing RBC aggregates. Compared to AA mice, SS mice showed significantly high in all 3 parameters, which were further increased upon TNFα injection by 51%, 59%, and 182%, respectively (see FIG. 15B).

### RBC Encapsulated P-Selectin Antibody Reduces Vaso-Occlusion During Steady State Conditions in SCD Mouse Liver

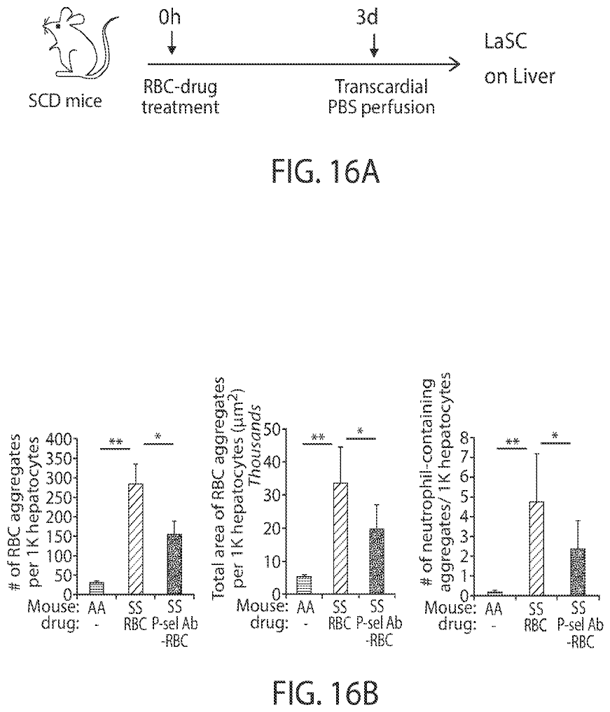

Three days after RBC-encapsulated P-selectin Ab injection (i.v.), the SCD mice were subjected to LaSC analysis for the quantification of RBC aggregates (>100 μm²) in livers (FIG. 16A). The # of RBC aggregates, the total area of RBC aggregates, and the # of neutrophil-containing RBC aggregates were reduced by 51%, 48%, and 52%, respectively, in SCD mice after RBC-encapsulated P-selectin Ab injection (FIG. 16B).

### RBC Encapsulated P-Selectin Antibody Reduces All Sizes of RBC Aggregates During Steady State Conditions in SCD Mouse Liver

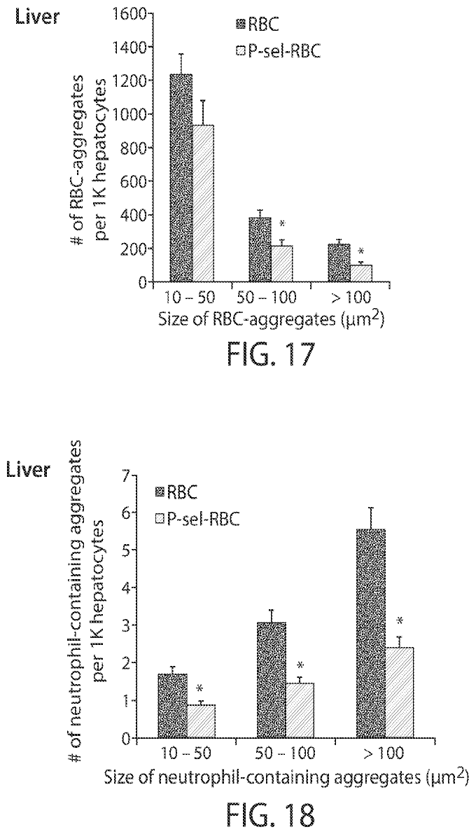

To test whether RBC encapsulated P-selectin antibody genuinely reduces RBC aggregates in all sizes, instead of merely shifting RBC aggregate sizes from large to small, SCD mice were subjected to LaSC analysis for the quantification of RBC aggregates, 3 days after RBC-encapsulated P-selectin Ab injection (i.v.) (FIGS. 17 and 18). The # of RBC aggregates were significantly reduced in both small (50-100 μm²) and large (>100 μm²) sizes (FIG. 17). Even the # of very small size RBC aggregates showed a tendency toward reduction. The # of neutrophil-containing RBC aggregates were also significantly reduced in very small (10-50 μm²), small (50-100 μm²), and large (>100 μm²) sizes (FIG. 18). Taken together, this data indicates that RBC encapsulated P-selectin antibody reduces RBC aggregates in all sizes.

### RBC Encapsulated P-Selectin Antibody Reduces Vaso-Occlusion During TNFα-Induced Inflammation in SCD Mouse Liver

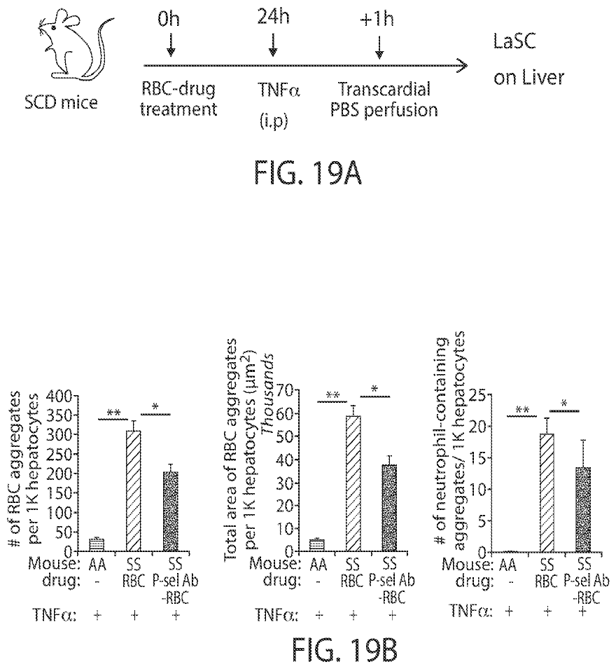

One day after RBC-encapsulated P-selectin Ab injection (i.v.), the SCD mice were subjected to TNFα injection (i.p., 1 h), followed by LaSC analysis for the quantification of RBC aggregates (>100 μm²) in livers (FIG. 19A). The # of RBC aggregates, the total area of RBC aggregates, and the # of neutrophil-containing RBC aggregates were reduced by 38%, 39%, and 28%, respectively, in TNFα injected SCD mice after RBC-encapsulated P-selectin Ab injection (FIG. 19B).

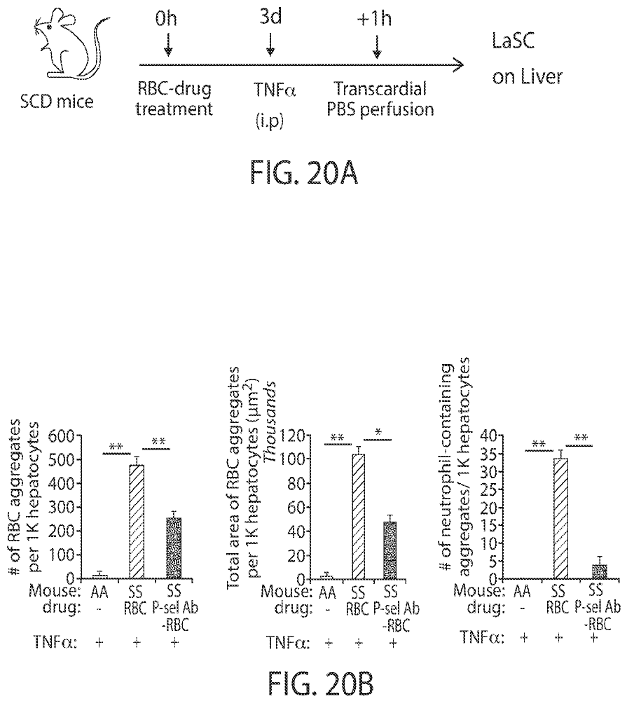

The effects of RBC-encapsulated P-selectin Abs were also examined 3 days after the injection in SCD mice upon TNFα injection. Three days after RBC-encapsulated P-selectin Ab injection (i.v.), the SCD mice were subjected to TNFα injection (i.p., 1 h), followed by LaSC analysis for the quantification of RBC aggregates (>100 μm²) in livers (FIG. 20A). The # of RBC aggregates, the total area of RBC aggregates, and the # of neutrophil-containing RBC aggregates were reduced by 48%, 55%, and 88%, respectively, in TNFα injected SCD mice after RBC-encapsulated P-selectin Ab injection (FIG. 20B), suggesting that prophylactic RBC-encapsulated P-selectin Ab treatment still works for a longer period (3 days) upon TNFα-injection.

### RBC Encapsulated Alteplase Reduces Vaso-Occlusion During Steady State Conditions in SCD Mouse Liver

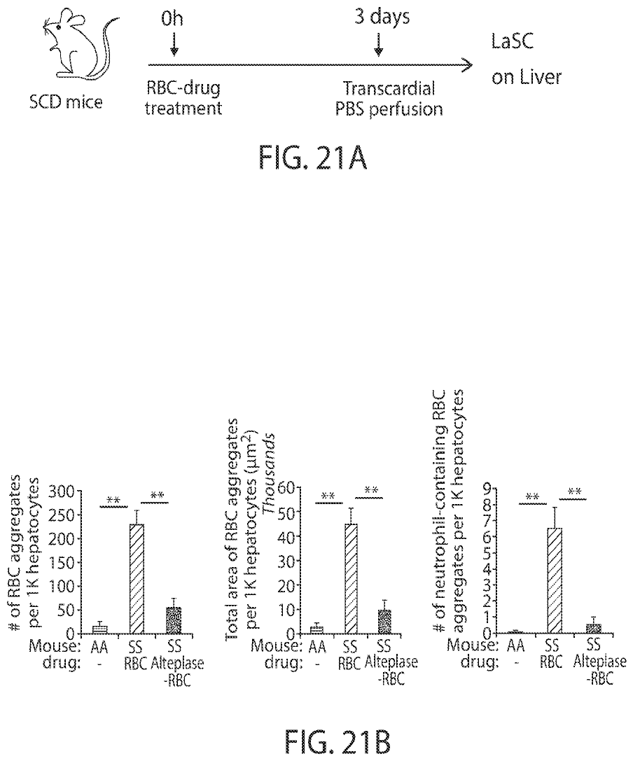

Alteplase, a recombinant tissue plasminogen activator, is a thrombolytic agent and converts plasminogen to plasmin in a blood clot. Three days after RBC-encapsulated Alteplase injection (i.v.), the SCD mice were subjected to LaSC analysis for the quantification of RBC aggregates (>100 μm²) in livers (FIG. 21A). The # of RBC aggregates, the total area of RBC aggregates, and the # of neutrophil-containing RBC aggregates were reduced by 82%, 83%, and 93%, respectively, in SCD mice after RBC-encapsulated Alteplase injection (FIG. 21B).

### RBC Encapsulated Alteplase Reduces Vaso-Occlusion During TNFα-Induced Inflammation in SCD Mouse Livers

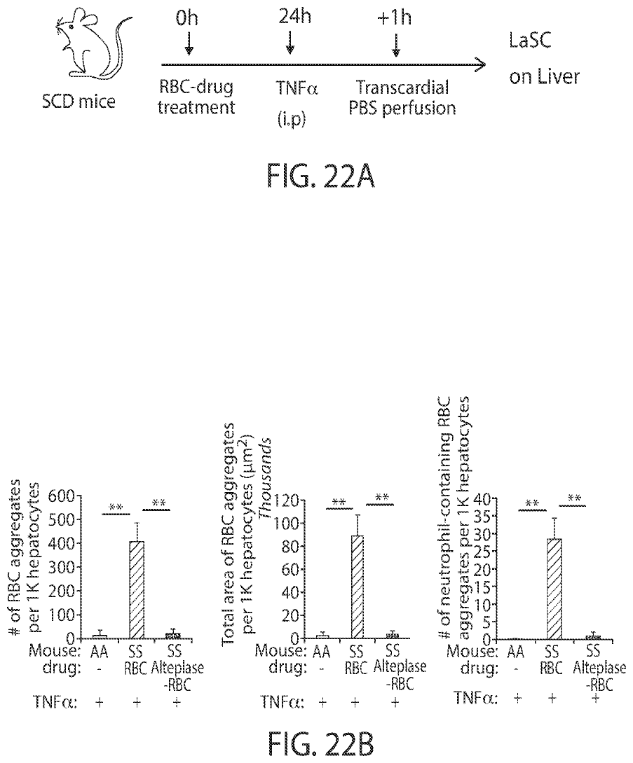

One day after RBC-encapsulated Alteplase injection (i.v.), the SCD mice were subjected to TNFα injection (i.p., 1 h), followed by LaSC analysis for the quantification of RBC aggregates (>100 μm²) in livers (FIG. 22A). The # of RBC aggregates, the total area of RBC aggregates, and the # of neutrophil-containing RBC aggregates were reduced by 98%, 99%, and 96%, respectively, in TNFα injected SCD mice after RBC-encapsulated Alteplase injection (FIG. 22B).

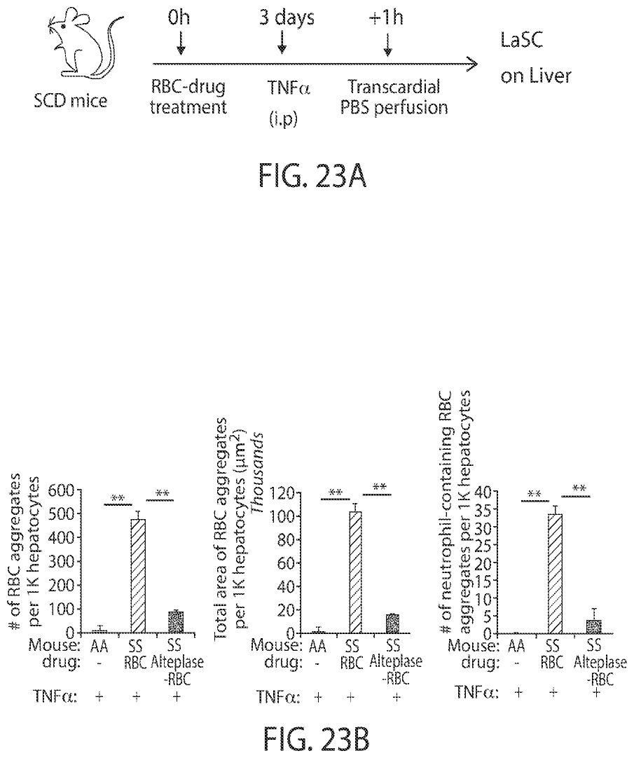

The effects of RBC-encapsulated Alteplase were also examined 3 days after the injection in TNFα injected SCD mice. Three days after RBC-encapsulated Alteplase injection (i.v.), the SCD mice were subjected to TNFα injection (i.p., 1 h), followed by LaSC analysis for the quantification of RBC aggregates (>100 μm²) in livers (FIG. 23A). The # of RBC aggregates, the total area of RBC aggregates, and the # of neutrophil-containing RBC aggregates were reduced by 83%, 86%, and 88%, respectively, in TNFα injected SCD mice after RBC-encapsulated Alteplase injection (FIG. 23B).

### RBC Encapsulated P-Selectin Antibody and Alteplase Reduce the Size of Enlarged Spleens and Recover Splenic B Cell Follicles in SCD Mice Under Steady State Conditions

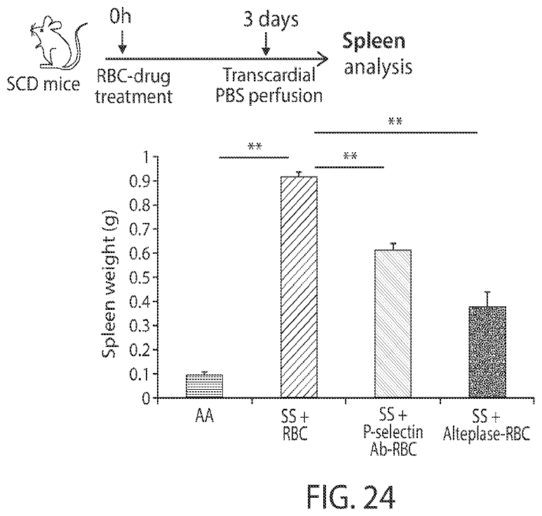

SCD or control mice were injected with indicated drug-encapsulated RBC (i.v.) for 3 days, followed by measurement of spleens as the schematic shows (FIG. 24). Spleen weights were plotted on a bar graph, showing that RBC-encapsulated P-selectin Ab and Alteplase reduce the size of enlarged spleen in SCD mice by 37% and 65%, respectively, under steady state conditions after 3 days (FIG. 24).

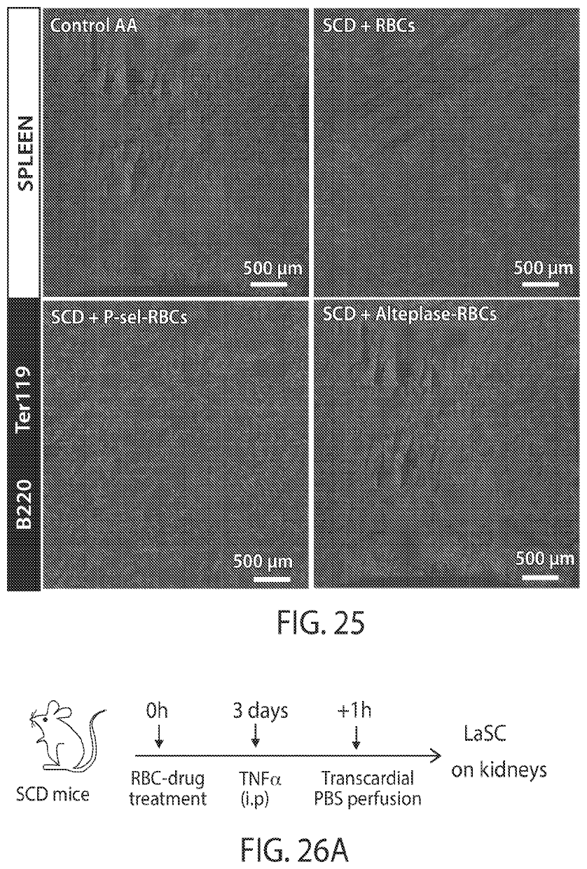

Due to expanded extramedullary erythropoiesis in SCD mouse spleen, SCD mice lose splenic B cell follicles, which play a key role in the immune function of spleen. From the LaSC analysis of SCD or control mice injected with drug-encapsulated RBC (i.v.) for 3 days, representative images are shown for spleens stained with immunofluorescent antibodies against Ter119 (for RBC marker) and B220 (for B cells) (FIG. 25). RBC-encapsulated P-selectin antibody and Alteplase restore B cell follicles in the spleens of SCD mice after 3 days.

### SCD Mouse Kidney Analysis Shows that RBC-Encapsulated P-Selectin Antibody and Alteplase Reduce Vaso-Occlusion During TNFα-Induced Inflammation

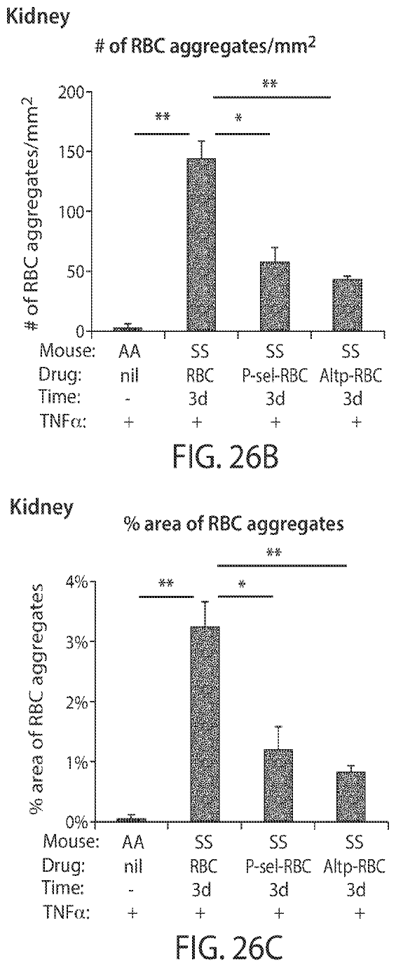

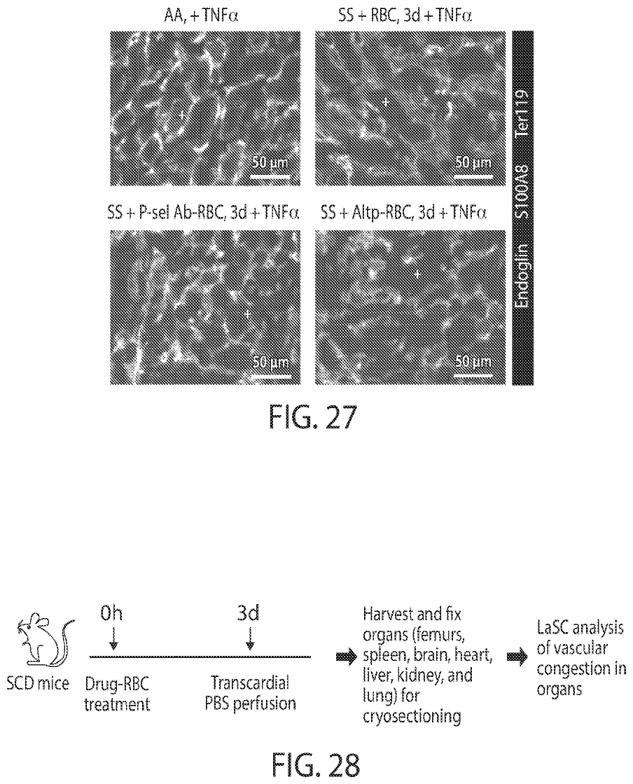

Three day after RBC-encapsulated drug injection (i.v.), the SCD mice were subjected to TNFα injection (i.p., 1 h), followed by LaSC analysis for the quantification of RBC aggregates (>100 μm²) in kidneys (FIG. 26A). The injections of RBC-encapsulated P-selectin antibody and Alteplase reduced # of RBC aggregates per 1 mm² kidney by 61% and 72%, respectively, in TNFα injected SCD mice after 3 days (FIG. 26B). In addition, the injections of RBC-encapsulated P-selectin antibody and Alteplase reduced the % area of RBC aggregates in the scanned kidney area by 64% and 76%, respectively, in TNFα injected SCD mice after 3 days (FIG. 26C). Representative images from LaSC analysis show the reduction of RBC aggregates in SCD mouse kidneys treated with RBC-encapsulated P-selectin antibody and Alteplase (FIG. 27).

### SCD Mouse Brain Analysis Shows That RBC-Encapsulated P-Selectin Antibody and Alteplase Reduce Vaso-Occlusion During Steady State Conditions

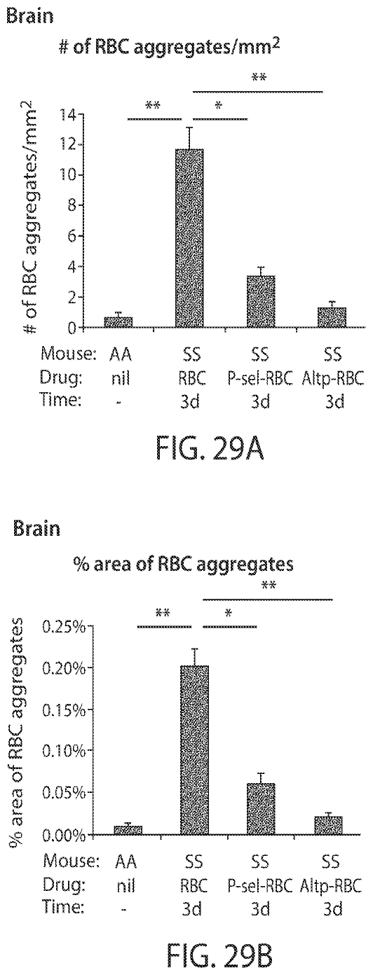

Three day after RBC-encapsulated drug injection (i.v.), the SCD mice were subjected to LaSC analysis for the quantification of RBC aggregates (>100 μm²) in brain (FIG. 28). The injections of RBC-encapsulated P-selectin antibody and Alteplase reduced # of RBC aggregates per 1 mm² brain by 76% and 94%, respectively, in SCD mice after 3 days (FIG. 29A). In addition, the injections of RBC-encapsulated P-selectin antibody and Alteplase reduced the % area of RBC aggregates in the scanned brain area by 74% and 94%, respectively, in SCD mice after 3 days (FIG. 29B).

### SCD Mouse Heart Analysis Shows That RBC-Encapsulated P-Selectin Antibody and Alteplase Reduce Vaso-Occlusion During Steady State Conditions

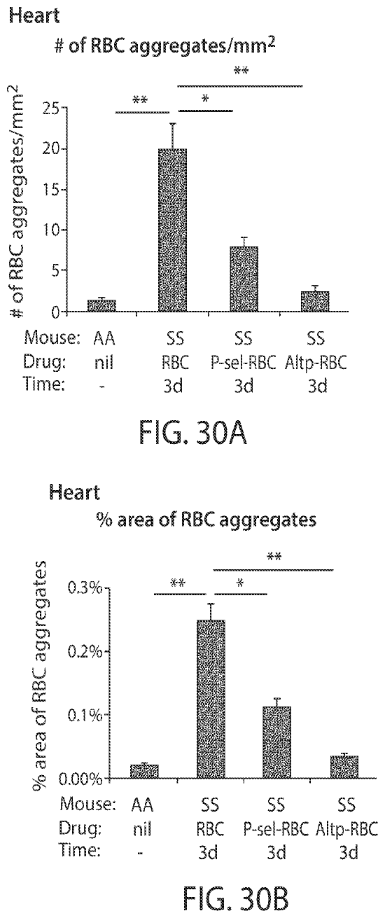

Three day after RBC-encapsulated drug injection (i.v.), the SCD mice were subjected to LaSC analysis for the quantification of RBC aggregates (>100 μm²) in heart (FIG. 28). The injections of RBC-encapsulated P-selectin antibody and Alteplase reduced # of RBC aggregates per 1 mm² heart by 65% and 94%, respectively, in SCD mice after 3 days (FIG. 30A). In addition, the injections of RBC-encapsulated P-selectin antibody and Alteplase reduced the % area of RBC aggregates in the scanned heart area by 60% and 94%, respectively, in SCD mice after 3 days (FIG. 30B).

### TNFα-Injected SCD Mouse Brain Analysis Shows That RBC-Encapsulated P-Selectin Antibody and Alteplase Reduce Vaso-Occlusion

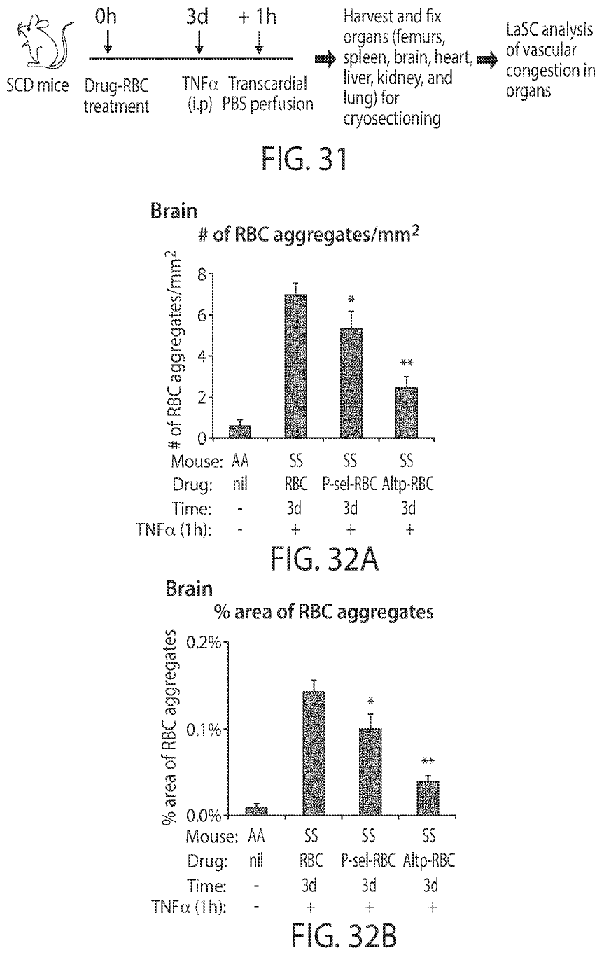

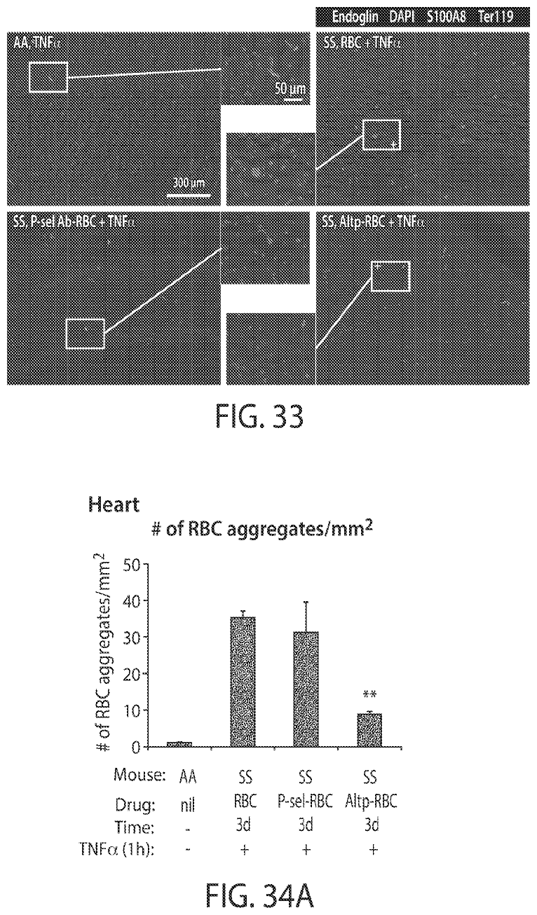

Three day after RBC-encapsulated drug injection (i.v.), the SCD mice were subjected to TNFα injection (i.p., 1 h), followed by LaSC analysis for the quantification of RBC aggregates (>100 μm²) in brain (FIG. 31). The injections of RBC-encapsulated P-selectin antibody and Alteplase reduced # of RBC aggregates per 1 mm² brain by 26% and 71%, respectively, in SCD mice after 3 days (FIG. 32A). In addition, the injections of RBC-encapsulated P-selectin antibody and Alteplase reduced the % area of RBC aggregates in the scanned brain area by 32% and 78%, respectively, in SCD mice after 3 days (FIG. 32B). Representative images from LaSC analysis show the reduction in the size and the frequency of RBC aggregates in SCD mouse brains treated with RBC-encapsulated P-selectin antibody and Alteplase (FIG. 33).

### TNFα-Injected SCD Mouse Heart Analysis Shows That RBC-Encapsulated Alteplase, But Not P-Selectin Antibody, Reduces Vaso-Occlusion

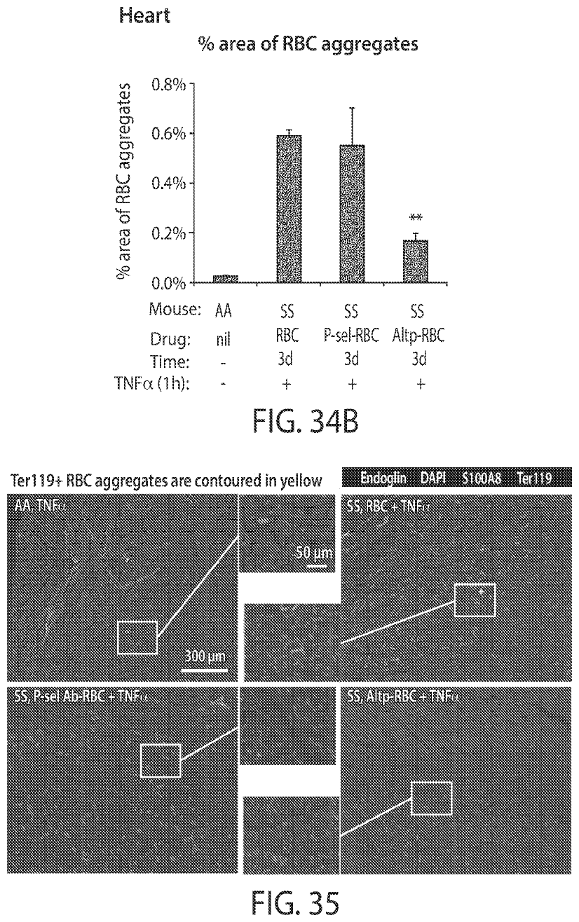

Three day after RBC-encapsulated drug injection (i.v.), the SCD mice were subjected to TNFα injection (i.p., 1 h), followed by LaSC analysis for the quantification of RBC aggregates (>100 μm²) in heart (FIG. 31). The injections of RBC-encapsulated Alteplase, but not P-selectin antibody, reduced # of RBC aggregates per 1 mm² heart by 78% in SCD mice after 3 days (FIG. 34A). In addition, the injections of RBC-encapsulated Alteplase, but not P-selectin antibody, reduced the % area of RBC aggregates in the scanned heart area by 74% in SCD mice after 3 days (FIG. 34B). Representative images from LaSC analysis show the reduction in the size and the frequency of RBC aggregates in SCD mouse heart treated with RBC-encapsulated Alteplase (FIG. 35).

### Widespread Intra-Sinusoidal Fibrin Clot Formation is Found in TNFα Injected SCD Mouse BM

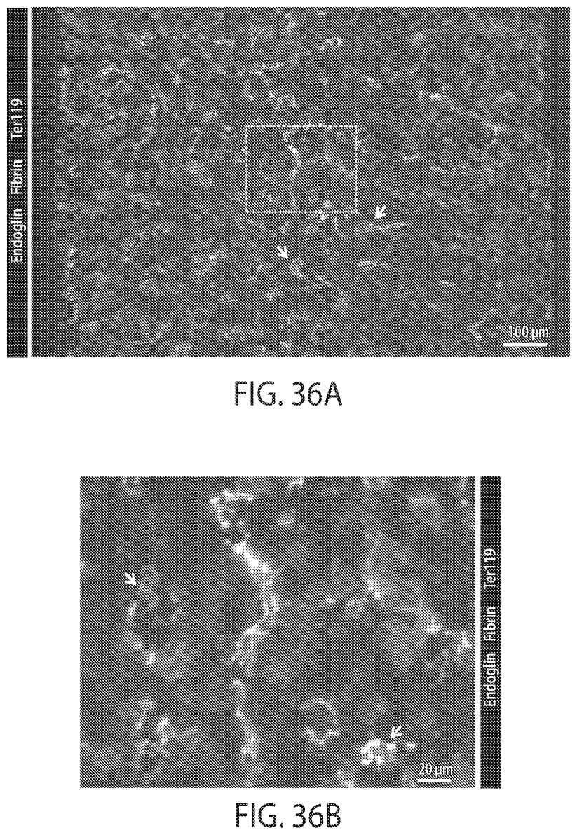

TNFα-injected SCD mouse BM were subjected to LaSC analysis after immunofluorescent staining with antibodies against fibrin/fibrinogen, endoglin, and Ter119, followed (FIG. 36A and 36B). LaSC images show widespread intra-sinusoidal fibrin clots in RBC aggregates of SCD mouse BM, suggesting that i) fibrin may mediate the hemolysis of transfused drug-loaded RBCs to deliver drugs in situ at the sites of RBC aggregates; ii) fibrin clots can also be targets for RBC-encapsulated thrombolytic drug.

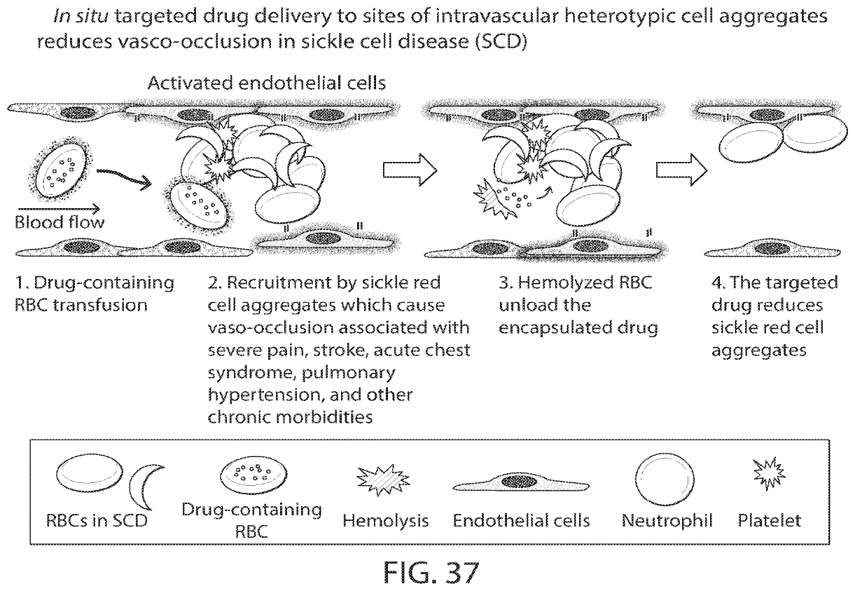

Taken together, in situ targeted drug delivery to sites of intravascular heterotypic aggregates reduces vaso-occlusion in SCD as shown in an illustration (FIG. 37). Transfused drug-containing RBCs flow in vessels and are recruited to RBC aggregates at vaso-occlusion sites. Transfused drug-containing RBCs are hemolyzed and unload in situ the encapsulated drug, which will reduce RBC aggregates/vaso-occlusion.

### Discussion

Approximately 100,000 people in the United States have sickle cell disease and every year over 300,000 babies are born worldwide with the condition. While gene therapy has recently been shown to cure the condition, its large expense means that it may not be widely available to the vast majority of people with SCD. Therefore, there is a need for a relatively inexpensive SCD treatment.

In the application, humanized sickle cell disease mice show that transfused RBCs containing encapsulated fluorescently labeled antibodies are recruited to heterocellular aggregates, undergo intravascular hemolysis and in situ release of the encapsulated antibodies which are detected bound to vascular endothelium. We have taken advantage of this unique red blood cell fate in SCD by encapsulating various drug candidates into red blood cells to target heterotypic aggregate formation.

Overall, transfusion of P-selectin antibody-loaded RBCs to SCD mice reduces RBC aggregates/congestion in major organs, such as liver, kidney, heart, brain, inhibiting SCD vaso-occlusion during steady state conditions. Additionally, TNFα-enhanced RBC aggregates was reduced in major organs, such as liver, kidney, heart, brain, inhibiting vaso-occlusion under stress conditions, such as inflammation and infection. P-selectin antibody can block the heterotypic adhesion among RBCs, platelets, and neutrophils, and the adhesion of those cells to activated vascular endothelial cells.

Additionally, transfusion of Alteplase-loaded RBCs to SCD mice reduces RBC aggregates in major organs, such as liver, spleen, kidney, heart, brain during steady state conditions. Additionally, TNFα-enhanced RBC aggregates were reduced in major organs, such as liver, spleen, kidney, heart, brain, inhibiting vaso-occlusion under stress conditions, such as inflammation and infection. As Alteplase works as a thrombolytic enzyme, and RBC-encapsulated Alteplase can be used to treat thrombosis in the brain and the heart.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

### REFERENCES

Alshaiban, A., V. Muralidharan-Chari, A. Nepo and S. A. Mousa (2016). “Modulation of Sickle Red Blood Cell Adhesion and its Associated Changes in Biomarkers by Sulfated Nonanticoagulant Heparin Derivative.” Clinical and Applied Thrombosis/Hemostasis 22(3): 230-238.
Ataga, K. I., A. Kutlar, J. Kanter, D. Liles, R. Cancado, J. Friedrisch, T. H. Guthrie, J. Knight-Madden, O. A. Alvarez, V. R. Gordeuk, S. Gualandro, M. P. Colella, W. R. Smith, S. A. Rollins, J. W. Stocker and R. P. Rother (2016). “Crizanlizumab for the Prevention of Pain Crises in Sickle Cell Disease.” New England Journal of Medicine 376(5): 429-439.
Barazia, A., J. Li, K. Kim, N. Shabrani and J. Cho (2015). “Hydroxyurea with AKT2 inhibition decreases vaso-occlusive events in sickle cell disease mice.” Blood 126(22): 2511-2517.
Belcher, J. D., C. Chen, J. Nguyen, F. Abdulla, P. Nguyen, M. Nguyen, N. M. Okeley, D. R. Benjamin, P. D. Senter and G. M. Vercellotti (2015). “The Fucosylation Inhibitor, 2-Fluorofucose, Inhibits Vaso-Occlusion, Leukocyte-Endothelium Interactions and NF-KB Activation in Transgenic Sickle Mice.” PLOS ONE 10(2): e0117772.
Belcher, J. D., C. Chen, J. Nguyen, L. Milbauer, F. Abdulla, A. I. Alayash, A. Smith, K. A. Nath, R. P. Hebbel and G. M. Vercellotti (2014). “Herne triggers TLR4 signaling leading to endothelial cell activation and vaso-occlusion in murine sickle cell disease.” Blood 123(3): 377-390.
Burnette, A. D., S. M. Nimjee, M. Batchvarova, R. Zennadi, M. J. Telen, J.-i. Nishimura and B. A. Sullenger (2011). “RNA Aptamer Therapy for Vaso-Occlusion in Sickle Cell Disease.” Nucleic Acid Therapeutics 21(4): 275-283.
Chaplin, H., Jr., M. C. Monroe, A. C. Malecek, L. K. Morgan, J. Michael and W. A. Murphy (1989). “Preliminary trial of minidose heparin prophylaxis for painful sickle cell crises.” East Afr Med J 66(9): 574-584.
Collen, D. and H. R. Lijnen (2005). “Thrombolytic agents.” Thromb Haemost 93(4): 627-630.
Desai, P. C., J. E. Brittain, S. K. Jones, A. McDonald, D. R. Wilson, R. Dominik, N. S. Key, L. V. Parise and K. I. Ataga (2013). “A pilot study of eptifibatide for treatment of acute pain episodes in sickle cell disease.” Thrombosis Research 132(3): 341-345.
Di Nisio, M., S. Middeldorp and H. R. Buller (2005). “Direct thrombin inhibitors.” N Engl J Med 353(10): 1028-1040.
Eke, F. U., A. Obamyonyi, N. N. Eke and E. A. Oyewo (2000). “An open comparative study of dispersible piroxicam versus soluble acetylsalicylic acid for the treatment of osteoarticular painful attack during sickle cell crisis.” Tropical Medicine & International Health 5(2): 81-84.
El-Beshlawy, A., E. Abd El Raouf, F. Mostafa, M. Talaat, H. Isma'eel, E. Aoun, A. V. Hoffbrand and A. Taher (2006). “Diastolic Dysfunction and Pulmonary Hypertension in Sickle Cell Anemia: Is There a Role for <i>L</i>-Carnitine Treatment?” Acta Haematologica 115(1-2): 91-96.
Field, J. J., K. I. Ataga, E. Majerus, C. A. Eaton, R. Mashal and D. G. Nathan (2013). “A Phase I Single Ascending Dose Study Of NKTT120 In Stable Adult Sickle Cell Patients.” Blood 122(21): 977-977.
Foroozesh, M., M. Hamidi, A. Zarrin, S. Mohammadi-Samani and H. Montaseri (2011). “Preparation and in-vitro characterization of tramadol-loaded carrier erythrocytes for long-term intravenous delivery.” J Pharm Pharmacol 63(3): 322-332.
Ginn, F. L., P. Hochstein and B. F. Trump (1969). “Membrane alterations in hemolysis: Internalization of plasmalemma induced by primaquine.” Science 164(3881): 843-845.
Gutsaeva, D. R., J. B. Parkerson, S. D. Yerigenahally, J. C. Kurz, R. G. Schaub, T. Ikuta and C. A. Head (2011). “Inhibition of cell adhesion by anti-P-selectin aptamer: a new potential therapeutic agent for sickle cell disease.” Blood 117(2): 727-735.
Hebbel, R. P., G. M. Vercellotti, B. S. Pace, A. N. Solovey, R. Kollander, C. F. Abanonu, J. Nguyen, J. V. Vineyard, J. D. Belcher, F. Abdulla, S. Osifuye, J. W. Eaton, R. J. Kelm, Jr and A. Slungaard (2010). “The HDAC inhibitors trichostatin A and suberoylanilide hydroxamic acid exhibit multiple modalities of benefit for the vascular pathobiology of sickle transgenic mice.” Blood 115(12): 2483-2490.
Heeney, M. M., C. C. Hoppe and D. C. Rees (2016). “Prasugrel for Sickle Cell Vaso-Occlusive Events.” N Engl J Med 375(2): 185-186.
Hoppe, C., E. Jacob, L. Styles, F. Kuypers, S. Larkin and E. Vichinsky (2017). “Simvastatin reduces vaso-occlusive pain in sickle cell anaemia: a pilot efficacy trial.” British Journal of Haematology 177(4): 620-629.
Jagadeeswaran, R., B. A. Vazquez, M. Thiruppathi, B. B. Ganesh, V. Ibanez, S. Cui, J. D. Engel, A. M. Diamond, R. E. Molokie, J. DeSimone, D. Lavelle and A. Rivers (2017). “Pharmacological inhibition of LSD1 and mTOR reduces mitochondrial retention and associated ROS levels in the red blood cells of sickle cell disease.” Exp Hematol 50: 46-52.
Kalish, B. T., A. Matte, I. Andolfo, A. lolascon, O. Weinberg, A. Ghigo, J. Cimino, A. Siciliano, E. Hirsch, E. Federti, M. Puder, C. Brugnara and L. De Franceschi (2015). “Dietary ω-3 fatty acids protect against vasculopathy in a transgenic mouse model of sickle cell disease.” Haematologica 100(7): 870-880.
Katusic, Z. S., L. V. d′Uscio and K. A. Nath (2009). “Vascular protection by tetrahydrobiopterin: progress and therapeutic prospects.” Trends in Pharmacological Sciences 30(1): 48-54.
KAUL, D. K., R. KOLLANDER, H. MAHASETH, X.-D. LIU, A. SOLOVEY, J. BELCHER, R. J. KELM Jr., G. M. VERCELLOTTI and R. P. HEBBEL (2006). “Robust Vascular Protective Effect of Hydroxamic Acid Derivatives in a Sickle Mouse Model of Inflammation.” Microcirculation 13(6): 489-497.
Koehl, B., P. Nivoit, W. El Nemer, O. Lenoir, P. Hermand, C. Pereira, V. Brousse, L. Guyonnet, G. Ghinatti, M. Benkerrou, Y. Colin, C. Le Van Kim and P.-L. Tharaux (2017). “The endothelin B receptor plays a crucial role in the adhesion of neutrophils to the endothelium in sickle cell disease.” Haematologica 102(7): 1161-1172.
Kwon, Y. M., H. S. Chung, C. Moon, J. Yockman, Y. J. Park, S. D. Gitlin, A. E. David and V. C. Yang (2009). “L-Asparaginase encapsulated intact erythrocytes for treatment of acute lymphoblastic leukemia (ALL).” J Control Release 139(3): 182-189.
Machado, R. F., R. J. Barst, N. A. Yovetich, K. L. Hassell, G. J. Kato, V. R. Gordeuk, J. S. R. Gibbs, J. A. Little, D. E. Schraufnagel, L. Krishnamurti, R. E. Girgis, C. R. Morris, E. B. Rosenzweig, D. B. Badesch, S. Lanzkron, O. Onyekwere, O. L. Castro, V. Sachdev, M. A. Waclawiw, R. Woolson, J. C. Goldsmith, M. T. Gladwin, o. b. o. t. w.-P. Investigators and Patients (2011). “Hospitalization for pain in patients with sickle cell disease treated with sildenafil for elevated TRV and low exercise capacity.” Blood 118(4): 855-864.
Mahaseth, H., G. M. Vercellotti, T. E. Welch, P. R. Bowlin, K. M. Sonbol, C. J. C. Hsia, L. Ma, J. C. Bischof, R. P. Hebbel and J. D. Belcher (2005). “Polynitroxyl albumin inhibits inflammation and vasoocclusion in transgenic sickle mice.” The Journal of Laboratory and Clinical Medicine 145(4): 204-211.
Martins, V. D., V. Manfredini, M. C. R. Peralba and M. S. Benfato (2009). “Alpha-lipoic acid modifies oxidative stress parameters in sickle cell trait subjects and sickle cell patients.” Clinical Nutrition 28(2): 192-197.
McArthur, J. G., T. Maciel, C. Chen, A. Fricot, D. Kobayashi, J. Nguyen, P. Nguyen, A. Parachova, F. Abdulla, G. M. Vercellotti, N. Svenstrup and J. D. Belcher (2016). “A Novel, Highly Potent and Selective PDE9 Inhibitor for the Treatment of Sickle Cell Disease.” Blood 128(22): 268-268.
Minniti, C. P., R. F. Machado, W. A. Coles, V. Sachdev, M. T. Gladwin and G. J. Kato (2009). “Endothelin receptor antagonists for pulmonary hypertension in adult patients with sickle cell disease.” British Journal of Haematology 147(5): 737-743.
Morris, C. R., F. A. Kuypers, L. Lavrisha, M. Ansari, N. Sweeters, M. Stewart, G. Gildengorin, L. Neumayr and E. P. Vichinsky (2013). “A randomized, placebo-controlled trial of arginine therapy for the treatment of children with sickle cell disease hospitalized with vaso-occlusive pain episodes.” Haematologica 98(9): 1375-1382.
Niihara, Y., N. M. Matsui, Y. M. Shen, D. A. Akiyama, C. S. Johnson, M. A. Sunga, J. Magpayo, S. H. Embury, V. K. Kalra, S. Ho Cho and K. R. Tanaka (2005). “L-Glutamine therapy reduces endothelial adhesion of sickle red blood cells to human umbilical vein endothelial cells.” BMC Hematology 5(1): 4.
Nur, E., D. P. Brandjes, T. Teerlink, H.-M. Otten, R. P. J. Oude Elferink, F. Muskiet, L. M. Evers, H. ten Cate, B. J. Biemond, A. J. Duits, J.-J. B. Schnog and C. s. g. on behalf of the (2012). “N-acetylcysteine reduces oxidative stress in sickle cell patients.” Annals of Hematology 91(7): 1097-1105.
Opene, M., J. Kurantsin-Mills, S. Husain and B. O. Ibe (2014). “Sickle Erythrocytes and Platelets Augment Lung Leukotriene Synthesis with Downregulation of Anti-Inflammatory Proteins: Relevance in the Pathology of the Acute Chest Syndrome.” Pulmonary Circulation 4(3): 482-495.
Park, S. Y., A. Matte, Y. Jung, J. Ryu, W. B. Anand, E. Y. Han, M. Liu, C. Carbone, D. Melisi, T. Nagasawa, J. J. Locascio, C. P. Lin, L. E. Silberstein and L. De Franceschi (2020). “Pathologic angiogenesis in the bone marrow of humanized sickle cell mice is reversed by blood transfusion.” Blood 135(23): 2071-2084.
Qari, M. H., S. K. Aljaouni, M. S. Alardawi, H. Fatani, F. M. Alsayes, P. Zografos, M. Alsaigh, A. Alalfi, M. Alamin, A. Gadi and S. A. Mousa (2007). “Reduction of painful vaso-occlusive crisis of sickle cell anaemia by tinzaparin in a double-blind randomized trial.” Thromb Haemost 98(2): 392-396.
Quinn, C. T., M. J. Stuart, K. Kesler, K. I. Ataga, W. C. Wang, L. Styles, K. Smith-Whitley, T. Wun, A. Raj, L. L. Hsu, S. Krishnan, F. A. Kuypers, Y. Setty, S. Rhee, N. S. Key, G. R. Buchanan and o. b. o. t. I. o. t. C. S. C. Centers (2011). “Tapered oral dexamethasone for the acute chest syndrome of sickle cell disease.” British Journal of Haematology 155(2): 263-267.
Sabaa, N., L. de Franceschi, P. Bonnin, Y. Castier, G. Malpeli, H. Debbabi, A. Galaup, M. Maier-Redelsperger, S. Vandermeersch, A. Scarpa, A. Janin, B. Levy, R. Girot, Y. Beuzard, C. Leboeuf, A. Henri, S. Germain, J.-C. Dussaule and P.-L. Tharaux (2008). “Endothelin receptor antagonism prevents hypoxia-induced mortality and morbidity in a mouse model of sickle-cell disease.” The Journal of Clinical Investigation 118(5): 1924-1933.
Schaid, T. R., Jr., J. Nguyen, C. Chen, F. Abdulla, T. Killeen, H. Nguyen, J. Edmund, M. A. Lindorfer, R. P. Taylor, A. P. Dalmasso, J. D. Belcher and G. M. Vercellotti (2016). “Complement Activation in a Murine Model of Sickle Cell Disease: Inhibition of Vaso-Occlusion By Blocking C5 Activation.” Blood 128(22): 158-158.
Semple, M. J., S. F. Al-Hasani, P. Kioy and G. F. Savidge (1984). “A Double-Blind Trial of Ticlopidine in Sickle Cell Disease.” Thromb Haemost 51(03): 303-306.
Shi, P. A., E. Choi, N. R. Chintagari, J. Nguyen, X. Guo, K. Yazdanbakhsh, N. Mohandas, A. I. Alayash, E. A. Manci, J. D. Belcher and G. M. Vercellotti (2016). “Sustained treatment of sickle cell mice with haptoglobin increases HO-1 and H-ferritin expression and decreases iron deposition in the kidney without improvement in kidney function.” British Journal of Haematology 175(4): 714-723.
Solovey, A., A. Somani, J. D. Belcher, L. Milbauer, L. Vincent, R. Pawlinski, K. A. Nath, R. J. Kelm, Jr., N. Mackman, M. G. O′Sullivan, K. Gupta, G. M. Vercellotti and R. P. Hebbel (2017). “A monocyte-TNF-endothelial activation axis in sickle transgenic mice: Therapeutic benefit from TNF blockade.” Am J Hematol 92(11): 1119-1130.
Telen, M. J., M. Batchvarova, S. Shan, P. H. Bovee-Geurts, R. Zennadi, A. Leitgeb, R. Brock and M. Lindgren (2016). “Sevuparin binds to multiple adhesive ligands and reduces sickle red blood cell-induced vaso-occlusion.” British Journal of Haematology 175(5): 935-948.
Telen, M. J., P. Malik and G. M. Vercellotti (2019). “Therapeutic strategies for sickle cell disease: towards a multi-agent approach.” Nat Rev Drug Discov 18(2): 139-158.
Telen, M. J., T. Wun, T. L. McCavit, L. M. De Castro, L. Krishnamurti, S. Lanzkron, L. L. Hsu, W. R. Smith, S. Rhee, J. L. Magnani and H. Thackray (2015). “Randomized phase 2 study of GMI-1070 in SCD: reduction in time to resolution of vaso-occlusive events and decreased opioid use.” Blood 125(17): 2656-2664.
Tsong, T. Y. (1991). “Electroporation of cell membranes.” Biophys J 60(2): 297-306.
Tsong, T. Y. and K. Kinosita, Jr. (1985). “Use of voltage pulses for the pore opening and drug loading, and the subsequent resealing of red blood cells.” Bibl Haematol (51): 108-114.
Valverde, Y., B. Benson, M. Gupta and K. Gupta (2016). “Spinal glial activation and oxidative stress are alleviated by treatment with curcumin or coenzyme Q in sickle mice.” Haematologica 101(2): e44-e47.
van Zuuren, E. J. and Z. Fedorowicz (2015). “Low-molecular-weight heparins for managing vaso-occlusive crises in people with sickle cell disease.” Cochrane Database of Systematic Reviews (12).
Vercellotti, G. M., P. Zhang, J. Nguyen, F. Abdulla, C. Chen, P. Nguyen, C. Nowotny, C. J. Steer, A. Smith and J. D. Belcher (2016). “Hepatic Overexpression of Hemopexin Inhibits Inflammation and Vascular Stasis in Murine Models of Sickle Cell Disease.” Molecular Medicine 22(1): 437-451.
Wood, K. C., R. P. Hebbel and D. N. Granger (2005). “Endothelial cell NADPH oxidase mediates the cerebral microvascular dysfunction in sickle cell transgenic mice.” The FASEB Journal 19(8): 989-991.
Zocchi, E., L. Guida, U. Benatti, M. Canepa, L. Borgiani, T. Zanin and A. De Flora (1987). “Hepatic or splenic targeting of carrier erythrocytes: a murine model.” Biotechnol Appl Biochem 9(5): 423-434.

What is claimed is:

1. A composition comprising a red blood cell (RBC) and a vasoocclusion-inhibiting agent wherein the vasoocclusion-inhibiting agent is encapsulated in the RBC.

2. The composition of claim 1, wherein the vasoocclusion-inhibiting agent is an anti-adhesion agent.

3. The composition of claim 2, wherein the anti-adhesion agent is an anti-P-selectin agent, Rivipansel, an anti-selectin aptamer, or an αvβ3 integrin inhibitor.

4. The composition of claim 3, wherein the anti-P-selectin agent is an anti-P-selectin antibody.

5. The composition of claim 4, wherein the anti-P-selectin antibody is a polyclonal antibody.

6. The composition of claim 4, wherein the anti-P-selectin antibody is a monoclonal antibody.

7. The composition of claim 4, wherein the anti-P-selectin antibody is Crizanlizumab.

8. The composition of claim 5, wherein the anti-P-selectin antibody is a monoclonal antibody.

9. The composition of claim 1, wherein the vasoocclusion-inhibiting agent is a tissue plasminogen activator.

10. The composition of claim 9, wherein the tissue plasminogen activator is Alteplase, Reteplase or Tenecteplase.

11. The composition of claim 1, wherein the vasoocclusion-inhibiting agent is an anti-coagulant agent.

12. The composition of claim 11, wherein the anti-coagulant agent is a direct thrombin inhibitor.

13. The composition of claim 12, wherein the direct thrombin inhibitor is Argatroban, Dabigatrin, or Lepirudin.

14. The composition of claim 1, wherein the vasoocclusion-inhibiting agent is an anti-inflammatory agent.

15. The composition of claim 14, wherein the anti-inflammatory agent is an endothelin antagonist.

16. The composition of claim 15, wherein the endothelin antagonist is Bosentan.

17. The composition of claim 1, wherein the vasoocclusion-inhibiting agent is a modulator of ischaemia-reperfusion and oxidative stress.

18. The composition of claim 1, wherein the vasoocclusion-inhibiting agent is an anti-platelet agent.

19. The composition of claim 1, wherein the vasoocclusion-inhibiting agent is an agent that counteracts free hemoglobin, heme, or iron.

20. The composition of claim 1, wherein the vasoocclusion-inhibiting agent is encapsulated in the RBC by ex vivo electroporation.

21. The composition of claim 1, wherein the vasoocclusion-inhibiting agent is encapsulated in the RBC by endocytosis methods.

22. The composition of claim 1, wherein the vasoocclusion-inhibiting agent is encapsulated in the RBC by cell-penetrating peptide (CPP)-mediated internalization.

23. The composition of any one of claims claim 1-22, wherein the RBC is an autologous RBC.

24. The composition of any one of claims claim 1-22, wherein the RBC is an allogenic RBC.

25. The composition of any one of claims claim 1-24, wherein the vasoocclusion-inhibiting agent is delivered to a site of a blood vessel occlusion.

26. The composition of any one of claims claim 1-25, wherein the vasoocclusion-inhibiting agent is released at the site of a blood vessel occlusion.

27. The composition of claim 25 or claim 26, wherein the blood vessel occlusion is caused by sickle cell disease.

28. The composition of any one of claims 25-27, wherein the blood vessel occlusion comprises a RBC aggregate.

29. The composition of any one of claims 25-27, wherein the blood vessel occlusion comprises a heterocellular aggregate.

30. The composition of claim 29, wherein the heterocellular aggregate comprises a RBC(s), a white blood cell(s) (WBC(s)), and a platelet(s).

31. The composition of any one of claims 1-30, further comprising a pharmaceutically acceptable carrier.

32. A method of treating a blood vessel occlusion in a subject, the method comprising administering to the subject in need thereof an effective amount of the composition of any one of claims 1-19.

33. The method of claim 32, wherein the blood vessel occlusion is caused by sickle cell disease.

34. The method of any of claim 32 or claim 33, wherein the composition is administered intravenously.

35. The method of any of claims 32-34, wherein the composition is administered once.

36. The method of any of claims 32-34, wherein the composition is administered repeatedly.

37. The method of any of claims 32-36, wherein the subject is a mammal.

38. The method of claim 37, wherein the mammal is a human.

39. The method of any of claims 32-38, wherein the effective amount of the composition reduces the size of the occlusion by 10%, 20%, 30%, 40%, 50%, 60%, 70%, 80%, 90%, or 100%.
